(19) United States
(12) Patent Application Publication
(10) Pub. No.: US 2026/0033880 A1
(43) Pub. Date: Feb. 5, 2026

(54) ELECTRICAL DEVICE

(71) Applicant: ATRIAN MEDICAL LIMITED, Galway (IE)

(72) Inventors: Stuart DEANE, Galway (IE); Barry O'BRIEN, Galway (IE); John REILLY, Galway (IE); Kenneth COFFEY, Galway (IE)

(73) Assignee: ATRIAN MEDICAL LIMITED, Galway (IE)

(21) Appl. No.: 18/997,391

(22) PCT Filed: Jul. 27, 2023
(86) PCT No.: PCT/EP2023/070819

§ 371 Date: Jan. 21, 2025

(30) Foreign Application Priority Data
Jul. 28, 2022 (EP) 22187491.0

(51) Int. Cl. A61B 18/00

(52) U.S. Cl. CPC A61B 18/00; A61B 2018/00577; A61B 2018/00642; A61B 2018/00672; A61B 2018/00702; A61B 2018/00761; A61B 2018/00767; A61B 2018/00827; A61B 2018/00875

(57) ABSTRACT

An apparatus for determining a voltage of electrical energy for subjecting to an adipose tissue, by comparing the calculated impedance value of the adjacent tissue with one or more previous calculated impedance values.

The invention relates to a fat sensing device and methods of using, for optimising the voltage for ablation. Further, the device may be for use on fat present on or near the heart, for example epicardial fat pads, and in particular identifying the interface between the adipose and muscle layers in order to optimise the voltage, used to ablate epicardial ganglionated plexi (GP) cells within the adipose layer.

### BACKGROUND

Many people suffer from abnormal heart conditions. Cardiac arrhythmias, such as atrial fibrillation, occurs when regions of the cardiac tissue abnormally conduct electric signals to adjacent tissue, thereby disrupting the normal cycle and causing asynchronous rhythm.

Procedures for treating arrhythmia include surgically disrupting the origin of the signals causing the arrhythmia, or disrupting the conducting pathway for such signals. By selectively ablating certain cardiac tissue by the application of energy, for example, it may be possible to stop or alter the signals from one portion of the heart to another. The ablation process helps to destroy abnormal signal-making cells or the pathway of the abnormal signal.

The ablation energy required, may be conveyed through electrodes, often the energy is passed from one electrode to another electrode, with the energy travelling through the target tissue to be ablated. Catheters, are often used to help position the electrode or at least direct the energy used for ablation. However, the procedure is still difficult, and time consuming, and has risks for the patient.

Epicardial ganglionated plexi (GP) can be ablated using pulsed electric fields as a means of treating atrial fibrillation. The pulsed electric field is often delivered via an electrode-tipped catheter positioned at specific epicardial fat pads, within which the GPs are located. The catheter delivers a fixed voltage to the tissue (fat) surface—this is typically in a bipolar manner, between adjacent positive and negative electrodes. The effectiveness of the Epicardial ganglionated plexi (GP) ablation is then dependent on achieving a sufficiently high electric field within the epicardial fat—the magnitude of the electric field depends on both the electrical properties and thickness of the fat, and the applied voltage. Specifically, fat has a high electrical impedance, and therefore as fat thickness increases, the magnitude of the resulting electric field drops off with increasing depth/distance from the point of contact with the electrodes.

Quantifying epicardial fat is usually referred to as thickness of the epicardial fat. The thickness is measured from the heart cardiac muscle tissue outwards from the heart.

Known methods to measure fat, for example, the thickness of epicardial fat, include imaging using ultrasound or X-rays. However, this often needs to be measured days before the planned surgery date, to allow highly trained medical staff, for example a radiographer, enough time to interpret the results. The imaging techniques of the prior art may also suffer that the amount of fat is calculated from that one particular view and this may not be consistent at other locations around the heart.

Previous devices and methods of measuring fat prior to surgery are in general time consuming to preform and analyse, that with, for example, the time delay between measurement and surgery, potentially means that the whole process is generally time consuming and inefficient.

In the absence of fat assessment, there is a risk that the wrong tissue will be ablated because the, for example, voltage used was too high. Or, in cases where the voltage use in the treatment, is too low, such that a less effective amount of voltage is used, potentially the cells are not ablated or inadequately ablated or the whole process takes much longer with unknowns to the effectiveness of the treatment.

The present invention aims to address some of these known problems with the prior art devices or methods.

### SUMMARY OF INVENTION DESCRIPTION

In a first aspect of the present invention there is provided an apparatus for, determining a voltage of electrical energy for subjecting to a tissue layer, for example wherein the tissue comprises adipose tissue, comprising:
at least a first electrode, and at least a second electrode, wherein the at least first electrode and the at least second electrode are spaced apart from each other, and wherein the at least first electrode and the at least second electrode comprise different polarity;
a controller configured for, or to, actuate at least a first electrode to emit a pulse of known voltage of electrical energy; and
a current sensor configured to, or for, measuring an electrical current, from the emitted pulse of electrical energy emitted from the said electrode and which said pulse extends through a portion of an adjacent tissue when in use; and,
a processor configured to or for; calculating an impedance value, from the known voltage of the pulse of emitted electrical energy and the electrical current; and for determining a voltage for the ablation of a tissue, by comparing the calculated impedance value of the adjacent tissue with one or more previous calculated impedance values.

In another aspect of the present invention there is provided an apparatus for determining a voltage of electrical energy for subjecting to an adipose tissue layer for ablation, comprising:
at least a first electrode, and at least a second electrode, wherein the at least first electrode and the at least second electrode are spaced apart from each other, and wherein the at least first electrode and the at least second electrode comprise different polarity;
a controller configured for actuating at least a first electrode to emit a plurality of pulses of known voltage of electrical energy; and
a current sensor configured for measuring an electrical current, for each emitted pulse of electrical energy emitted from the said electrode when said pulse extends through a portion of an adjacent tissue completing the electrical circuit; and,
a processor configured for; calculating an impedance value, from the known voltage of the said pulse of emitted electrical energy and the measured electrical current; and for determining a voltage for subjecting to an adjacent tissue layer for ablation, by comparing the calculated impedance value of a pulse of electrical energy of a known voltage with one or more previous calculated impedance values calculated from previously emitted pulses of electrical energy of a different voltage value.

In some embodiments, the an adjacent tissue comprises adipose tissue. Meaning that in use the apparatus of the present invention or the at least first electrode, is adjacent a tissue, an adjacent tissue, wherein the said adjacent tissue may comprise adipose tissue, for example cardiac adipose tissue, or an adipose layer, which comprises adipose tissue. Thus the emitted pulse of electrical energy may travel through a portion of the an adjacent tissue. The tissue completes the circuit of electricity, preferably between two electrodes for example between the at least first electrode and the at least second electrode.

In some embodiments, the current sensor is further configured to, or for, measuring an electrical current, from the emitted pulse of electrical energy emitted from the said electrode and which said emitted pulse of electrical energy extends through a portion of an adjacent tissue when the apparatus is in use, preferably wherein the adjacent tissue comprises adipose tissue. Adipose tissue, or layer (comprising adipose tissue), has a high impedance value compared to some other tissues, for example, muscle tissue. Usually adipose tissue has a higher impedance value than muscle tissue. Therefore the invention and apparatus of the invention, by measuring and comparing the impedance value in the an adjacent tissue can determine if the encompassed tissue of the electrical field or electrical energy, is only adipose tissue, or both adipose tissue and another tissue, for example, muscle tissue due to the change in impedance, or rate of change in impedance in relation to the voltage of the pulses of electrical energy. Tissue comprising even a portion of muscle tissue may have a lower impedance value than tissue that is only, or nearly only adipose tissue. The invention may also be for determining a safe voltage for use to subject to an adjacent, for example cardiac adipose tissue, in order to ablate target cells, for example ganglionated plexi cells, within the adipose tissue but that an adjacent tissue may comprise both adipose tissue and other non-adipose tissue, for example, underlying muscle tissue. In some embodiments, the adjacent tissue comprises both adipose tissue and muscle tissue.

In some embodiments the adjacent tissue is skin. In some embodiment the adjacent tissue comprises skin. In some embodiments it is foreseen that the two electrodes can be placed on or adjacent to skin of a subject, to measure the underlying tissue.

In some embodiments the tissue is cut tissue, for example cut butcher's meat like beef or pork, or cadaver tissue. In some embodiments the an adjacent tissue comprises cadaver tissue. In some embodiments the an adjacent tissue comprises cut tissue, for example cut butcher's joints like beef or pork, or cadaver tissue. In some embodiments it is envisaged that the two electrodes are adjacent a piece of cut tissue, for example cadaver tissue, to measure the fat content, or thickness of the adipose or fat tissue layer. Such results may be used to generate computer modelling of a patient subject to predict suitable voltages to be used for ablation of a target tissue within the patient's tissue. Other means of producing computer modelling, using the present invention, to predict adipose tissue thickness and suitable voltage of ablation of a target tissue can be foreseen.

In some embodiments, when in use, the an adjacent tissue completes an electrical circuit between the spaced apart electrodes. In some embodiments both the first and second electrodes are positioned adjacent a tissue. The adjacent tissue acts as an electron bridge to complete an electrical circuit between the electrodes when the pulse of electrical energy is emitted. In use of the apparatus in a position wherein the at least first electrode is adjacent a tissue, for example an adipose tissue or adipose layer, the electrical circuit is complete between the at least, first and second electrodes. Preferably both the at least first and the at least second electrodes are adjacent to the an adjacent tissue, such that the an adjacent tissue completes the electric circuit between the at least first electrode and the at least second electrode. When, in use, the electrical circuit is completed by positioning the electrodes to a side of the adjacent tissue, current from the pulse of electrical energy flows through a portion of the tissue adjacent to the electrodes.

Ideally the apparatus is for determining a voltage of electrical pulses for subjecting to an adipose layer wherein the voltage of electrical energy pulses may ablate target tissues, or cells, within the adipose layer, for example, may ablate ganglionated plexi cells, and as a further example wherein the target tissues or cells are epicardial ganglionated plexi cells within the adipose layer, for example, of epicardial fat pad.

The processor of the present invention is able, using the known voltage and measured current, to calculate an impedance value of the adjacent tissue, or portion thereof, by using Ohm's Law equation. This impedance value need not be an exact value but merely a relative value for when the calculated impedance value is compared to previously calculated impedance values. For example, when compared to previously calculated impedance values of earlier emitted pulses of electrical energy. In some embodiments, the calculated impedance value of the portion of adjacent tissue comprises a relative impedance value.

In some embodiments, the impedance value calculated is an impedance value of a portion of the adjacent tissue. This may be a relative value of impedance or impedance for the portion of adjacent tissue, for example, adjacent adipose tissue or adipose and muscle tissue.

In some embodiments the processor is further configured for, or comprises a configuration, to determine the voltage for subjecting to an adipose layer when the calculated impedance value matches a known previously calculated impedance value, calibrated for the apparatus, indicating a voltage, for example an optimal voltage, for ablation of the tissue thickness with that impedance value.

In some embodiments, the processor is further configured for, or comprises a configuration, to, determine the voltage for subjecting to an adipose layer when the calculated impedance value is different from the previous calculated impedance value, from a previous emitted pulse of electrical energy.

The actual thickness of the adipose layer is not required as the values are relative, but advantageously the invention may obtain, practically, real time information of the tissue, for example, for ablation to determine, for example, a suitable or optimal, voltage for subjecting to an adipose layer, for example, ablation of cells within an adipose layer. This may include a voltage for subjecting to an adipose layer or ablation of target cells within the adipose layer, that may potentially be less harmful than other greater voltages. This may include a voltage that is more effective than pulses of less voltage that potentially cannot penetrate enough of the fat layer for effective ablation of target cells within the adipose layer.

Advantageously the invention may determine a suitable voltage for subjecting to a tissue, for example adipose tissue, ablation of cells within the adipose tissue, without the need to determine the exact thickness. This also avoids any issue that adipose tissue of the same thickness may have different impedance, and the present invention has the benefit that it may allow determination of a suitable voltage for ablation for tissue of the same thickness but with different impedance or resistance values. If one tried to apply the same voltage for ablation to all tissue of the same thickness varying results may result, sometimes not enough of the target tissue will be subjected to electrical energy to be really effective and sometime it may result that the electrical energy extends to underlying tissue like muscle, causing damage to the muscle tissue.

In some embodiments determining a voltage for subjecting to an adipose layer, comprises determining an optimal voltage for subjecting to an adipose layer, for example, of an adjacent adipose tissue. Ideally, the pulse of electrical energy used for subjecting to an adipose layer will penetrate through a portion of the tissue, from the surface of an electrode in position adjacent to the tissue, in electrical contact with the electrode in position, including in a perpendicular direction into the tissue, away in direction from the electrode. In embodiments where the invention is to treat epicardial fat pads the pulse of electrical energy ideally will penetrate from the surface of the tissue in electrical contact with the electrode, towards the heart muscle. Ideally, the pulse of electrical energy used for subjecting to an adipose layer will penetrate through the layer of adipose tissue but not penetrate the muscle tissue of the heart. Ideally, the pulse of electrical energy will be of a sufficient voltage to penetrate through the adipose tissue to treat or ablate the target cells within the adipose tissue, for example, epicardial ganglionated plexi GP within the adipose tissue. In some embodiments, the pulse of electrical energy will be of a sufficient voltage to penetrate through the adipose tissue to treat or ablate the target tissue within the adipose tissue, for example, epicardial ganglionated plexi GP within the adipose tissue. In some embodiments, this may be enough voltage to penetrate through skin and other tissue, for example adipose tissue, to reach the target cells for ablation. The target cells for ablation may be within adipose tissue and therefore a portion of the adipose tissue itself may be subjected to electrical energy.

In some embodiments the determined voltage for subjecting to an adipose tissue layer comprises a voltage for ablation of the target cells within the target tissue for example, ganglionated plexi cells (GP), for example within an epicardial fat pad.

In some embodiments, the determined voltage for subjecting to an adipose tissue layer, comprises wherein the pulse of determined voltage extends through, at least 75 percent of the thickness of the adipose tissue layer. In specific embodiment the adjacent tissue layer comprises adipose tissue. In specific embodiments, when in use, the adjacent tissue is in electrical contact with the at least first and at least second electrodes,

In some embodiments the an adjacent tissue in use comprises adipose tissue.

In some embodiments, the determined voltage for subjecting to an adjacent adipose layer, comprises wherein the pulse of determined voltage extends through, at least 75 percent of the thickness of the adjacent adipose layer.

Extending through a percentage of the thickness of the adipose tissue layer as herein described and used, means that this would be measured perpendicularly from the electrode, as a percentage of the total thickness of the adipose layer.

In some embodiments, the determined voltage for subjecting to an adipose tissue layer, comprises wherein the pulse of determined voltage extends through at least 85 percent of the thickness of the adipose tissue layer. In some embodiments, the determined voltage for subjecting to an adipose tissue layer, comprises wherein the pulse of determined voltage extends through at least 95 percent of the thickness of the adipose tissue layer. In some embodiments, the determined voltage for subjecting to an adipose layer, comprises wherein the pulse of determined voltage extends through at least 99 percent of the thickness of the adipose layer.

In some embodiments, the determined voltage for subjecting to an adipose layer, comprises wherein the pulse of determined voltage extends through 75 to 99 percent of the thickness of the adipose layer. In some embodiments, the determined voltage for subjecting to an adipose layer, comprises wherein the pulse of determined voltage extends through 85 to 99 percent of the thickness of the adipose layer. In some embodiments, the determined voltage for subjecting to an adipose layer, comprises wherein the pulse of determined voltage extends through 95 to 99 percent of the thickness of the adipose layer. In some embodiments, the determined voltage for subjecting to an adipose layer, comprises wherein the pulse of determined voltage extends through 75 to 95 percent of the thickness of the adipose layer. In some embodiments, the determined voltage for subjecting to an adipose layer, comprises wherein the pulse of determined voltage extends through 85 to 95 percent of the thickness of the adipose layer. In some embodiments, the determined voltage for subjecting to an adipose layer, comprises wherein the pulse of determined voltage extends through 90 to 95 percent of the thickness of the adipose layer. These percentages of the thickness of the adipose layer are from the electrode, for example, perpendicularly from the electrode and for example towards the heart of a patient with the apparatus is positioned for use. In some embodiments, the determined voltage comprises wherein the percentage that the determined voltage for subjecting to an adipose layer extends through the adipose tissue is limited to 90, or 95 or 99 percent, of the thickness of the adipose tissue extending perpendicular from the electrode.

Advantageously the determined voltage for subjecting to an adipose layer, for a pulse of electrical energy used for ablation for example of target cells within an adipose layer or tissue, may penetrate, or extend through, a significant portion of the thickness of the adipose layer or tissue, from the electrode towards, for example, the centre of the heart or any muscle tissue underlying the fatty layer, without extending beyond, or penetrating completely through, the adipose layer or tissue.

When the tissue, for example adipose tissue, is subject to electrical energy, if the voltage of pulse of electrical energy is too high, this increases the risk that, for example underlying muscle tissue, will be subjected to electrical energy and may damage the muscle tissue. A higher voltage presents more risk that the electrical field will extend beyond the fat or adipose layer, and to other tissue, for example muscle tissue.

When the tissue, for example adipose tissue, is subject to electrical energy, if the voltage of the pulse of electrical energy is too low, this increases the risk that the electrical energy may not penetrate all the adipose tissue and therefore be ineffective at ablation to all, or nearly all, the targeted cells within the adipose tissue.

Advantageously, the present invention helps determine a suitable or, an optimal, voltage that is effective at ablation of the targeted tissue, for example the ganglionated plexi cells within adipose tissue, while reducing the risk of damaging near non-targeted, non-adipose tissue, for example muscle tissue, and in particular cardiac muscle tissue. The person skilled in the art will understand that a voltage, or an optimal voltage, need not be an exact single optimum voltage to be effective, and, that the determined voltage for subjecting to an adipose layer, or ablation of target cells within the adipose tissue, of the present invention works is a voltage or voltages that helps to give effective penetration of the electrical energy through the adipose layer and/or ablation results of the target cells, for example, ganglionated plexi cells (GP), within an adipose tissue, while reducing risks of damaging other near tissue.

In some embodiments, the processor is further configured for, or comprises a configuration, to determine an optimal voltage for effective penetration through the adipose layer and/or the ablation of the target cells within the adjacent adipose tissue. When the voltage is used as electrical energy subjected to the adipose layer and for ablation of the target cells.

In some specific embodiments, the adipose tissue comprises epicardial ganglionated plexi cells (GP). In specific embodiments, the adipose tissue comprises epicardial fat tissue. In some embodiments, the epicardial ganglionated plexi cells (GP) within the adipose tissue may be the target cells for ablation.

In some embodiments, there further comprises a plurality of electrodes. Having a second electrode especially an electrode of a different polarity helps direct the flow direction of current, or a user will know the general direction of current from one electrode to the other. A greater number of electrodes may enable a greater area of tissue to be measured, or ablated at one time.

Different embodiments of the invention may have different number of electrodes. In some embodiments having two or more electrodes, the second electrode comprises a different polarity from the first electrode. In some embodiments, a plurality of electrodes comprise at least one positive charged electrode and at least one negative charged electrode. In some embodiments, a plurality of electrodes comprise at least one anode electrode and at least one cathode electrode. In some embodiments, the electrodes may work as pairs, for example, of differently changing pairs of electrodes. In some embodiments, in each pair of electrodes there will be one positive charged electrode and one negative charged electrode. In some embodiments, a pair of electrodes that work together may have the same charge and work with: another electrode; or pair of electrodes; or a plurality of electrodes; of an opposite charge.

In some embodiments, the controller is further configured for, or comprises a configuration, to actuate emitting a pulse of electrical energy of a known voltage, independently from any one or more of the following group: the at least first electrode; the at least second electrode; and any subsequent electrode. Advantageously this enables a wide range of different electrical fields in the area of the electrodes of the present invention.

In some embodiments, the controller is further configured for, or comprises a configuration, to actuate at least one electrode that at least a first pulse of electrical energy and at least a second pulse of electrical energy are of a different voltage value. The different voltage values of the pulses of electrical energy may result in different electrical field strength and may give a different impedance value, that the processor may be configured for, or comprise a configuration, to determine. The processor maybe configured for, or comprise a configuration, to calculate the impedance from the known voltage value and the generated electrical current.

In some embodiments, the at least second pulse of electrical energy comprises, or is of, a greater voltage value than the at least first, or previous, pulse of electrical energy.

In some embodiments the controller is further configured for, or comprises a configuration, to actuate the electrode to emit a number, or a plurality, of pulses of electrical energy wherein the number of pulses is n; and, each pulse has a known voltage V. The specific voltage V to the specific pulse n maybe Vn.

In some embodiments, the controller is further configured, or comprises a configuration, such that each consecutive pulse increasing in number n, of a plurality of consecutive pulses, has an increasing voltage V. In some embodiments, the current sensor is configured, or comprises a configuration, to measure the electrical current of each pulse n, for example of a plurality of consecutive pulses.

In some embodiments, the voltage value of the pulse increases in steps of 1 or 2 volts, for example of a plurality of consecutive pulses. In specific embodiments, the voltage value of the pulse increase in steps of any one or more of the following groups: 0.1 volts; 0.5 volts 1 volt; 2 volts; 5 volts; 10 volts; 20 volts; 50 volts; 100 volts and 1000 volts. Advantageously the voltage change can be: large for quick rough estimation of the thickness of the adipose tissue; or small for fine-tuning to find accurate thickness of the thickness of the adipose tissue. In alternative embodiments the voltage value may start large and decrease in value; or indeed alternate between increasing and decreasing in the voltage value. In specific embodiments the processor may further comprise a configuration, for using the measured and calculated information obtained to choose particular voltages for pulses of electrical energy. This may enable the processor to react to the information obtained to quickly fine-tune to the determined voltage for ablation of the adipose tissue. In specific embodiments, the processor may further comprise a configuration that uses measured and calculated information during the ablation mode of the apparatus to be able to inform a user of change; or change the voltage of the pulse of electrical energy, or other parameters, during the ablation mode of the apparatus.

In some embodiments, the current sensor is configured for, or comprises a configuration, to convey, information that the current sensor measures, to the processor.

In some embodiments, the processor is further configured for, or comprises a configuration: to calculate an impedance value Z, from the known voltage V and the electrical current I for each pulse n:
and to monitor the impedance values; and to comparing the impedance value Z for each pulse n with the impedance value to an earlier emitted pulse, for example, N minus 1. In specific embodiments, the processor is further configured for, or comprises a configuration, to compare the impedance value of one pulse with all, or a selected group of, previous pulses, for example, wherein the pulses comprise the same known voltage or the pulses comprise different voltages.

In some embodiments, the processor is further configured for, or comprises a further configuration, to signal the controller upon detection that, the impedance value Z of the pulse n, decreases, or is less than, in value compared to the impedance value Z of the earlier emitted pulse, for example N minus 1.

In some embodiments, the processor is further configured for, or comprises a further configuration, to signal the controller upon detection that, the impedance value Z of the pulse n, increases, or changes, in value compared to the impedance value Z of the earlier emitted pulse, for example N minus 1.

In some embodiments the change in impedance value, or rate of change in impedance value in relation to voltage, comprises a 10 percent, or 15 percent, or 20 percent change, for example decrease, compared to the previously emitted pulse of electrical energy.

In some embodiments the processor is further configured for, to signal the controller upon detection that, that the impedance value of a pulse of electrical energy, decreases, or is less than, in value compared to the impedance value of an, or the earlier emitted pulse.

In some embodiments the processor is further configured for, to signal the controller upon detection that, that the impedance value of a pulse of electrical energy, decreases, or is less than, in value by at least 10 percent, or at least 15 percent or at least 20 percent compared to the impedance value of an, or the earlier emitted pulse.

In some embodiments the processor is further configured for, to signal the controller upon detection that, that the rate of change of impedance in relation to voltage a pulse of electrical energy, decreases, or is less than, in value compared to the rate of impedance in relation to voltage of an, or the earlier emitted pulse.

In some embodiments the processor is further configured for, to signal the controller upon detection that, that the impedance value of a pulse of electrical energy, decreases, or is less than, in value by at least 10 percent, or at least 15 percent or at least 20 percent compared to the rate of change of impedance in relation to voltage of an, or the earlier emitted pulse.

In some embodiments the processor is configured, or is further configured, to calculate the rate of change of the impedance value in relation to the voltage for the pulses of electrical energy. This is advantageous where the adjacent tissue does not drop in value of impedance to increasing voltage but that there is a change in the rate of change of the impedance value, possibly due to the electrical energy from the pulse extending through the adipose tissue to another tissue, for example, muscle that has a lower impedance than adipose tissue. Advantageously the present invention is sensitive enough to determine adipose to for example muscle interface, to determine a suitable voltage value for ablation to subject to adipose tissue without extending through to the underlying muscle tissue.

In some embodiments the controller is configured for actuating at least a first electrode to emit at least two, or at least three, or at least four, or at least five, or at least six, pulses of known voltage of electrical energy. Advantageously the rate of change in impedance, in relation to voltage is a comparison to the rate of change of impedance at another time point. The rate of change of impedance in relation to voltage can be calculated for each pulse of a known voltage when compared to the impedance value of an earlier pulse of known voltage, over time. In this way each pulse can have its own rate of change of impedance value compared to the earlier pulse emitted.

In some embodiments the processor is configured to determine the rate of change of impedance of each pulse. The person skilled in the art would understand how a processor could determine this from the known voltage values of the pulses of electrical energy, measuring the current to determine the impedance value and knowing the time difference between pulses.

In some embodiments the processor is further configured for, to signal the controller, or user, or both user and controller, upon detection that, the impedance value Z of the pulse n, decreases, or is less than, in value compared to the impedance value Z of an, or the earlier emitted pulse.

Additionally or alternatively in some alternative embodiments the processor may be configured to detect when the impedance value increases, or the rate of change of impedance value in relation to voltage increase. The person skilled in the art would understand that the pulses of the electrical energy could be initially be set high and consecutive pulses decrease in voltage thus detecting when the impedance or rate of impedance increases may be important.

In some embodiments the processor is further configured for, to signal the controller, or user, or both controller and user, upon detection that, the rate of change of the impedance value of the said pulse n compared to the voltage value, decreases, or is less than, in value compared to the rate of change of the impedance value of an, or the earlier emitted pulse. A decrease in the rate of change of impedance value in relation to voltage may indicate that the electrical energy has extended to, in the electrical field, a different tissue, for example muscle tissue, of a lower impedance or resistance value generally.

In some embodiments the processor is configured for, to signal the controller, or a user, or both a user and controller, that the determined voltage for subjecting to an adjacent, for example adipose, tissue layer for ablation, is the voltage V of the earlier emitted pulse. The invention by incremental steps of increasing voltage may determine a voltage suitable for ablation when the highest voltage used of a pulse indicates by the impedance value or rate of change of the impedance value, for example decreasing in value from a previously emitted pulse, that the electrical field has reached the underlying muscle tissue. Therefore the most effective voltage for ablation, but without penetrating through to underlying muscle tissue but be the voltage value of the immediately earlier emitted pulse of electrical energy.

In some embodiments the controller is configured for, that when signalled that the impedance Z for a pulse from the plurality of consecutive pulses, decreases in value, or is less than in value, compared to the impedance value with a, or the, previous emitted pulse, or the rate of change of the impedance value compared to the change of the voltage, decreases, or is less than in value to previously calculated rate of change of impedance value, the controller is configured to stop increasing the voltage value of the pulses of electrical energy to be emitted. This gives the benefit that potentially harmful energy pulses are not emitted.

In some embodiments the controller is further configured for, that when signalled that the impedance Z of a pulse from the plurality of pulses, decreases in value, or is less than, compared to the impedance value of a, or the previous emitted pulse, or the rate of change of the impedance value compare to the change of voltage, of pulses, decreases, or is less in value compared to previous values of the rate of change of impedance, the controller is configured to indicate to a user, or the controller, or both the controller and a user, that the apparatus is ready for ablation mode. This gives the benefit that the device is ready for use for ablation of target cells in an adjacent tissue with a safe but effective voltage value for the tested adjacent tissue.

In some embodiments the controller is further configured for, that when signalled that the impedance Z has decreased in value, or is less than in value to that determined for a previous emitted pulse from the plurality of pulses comprising increasing voltage, or the rate of change of impedance value in relation to the change of voltage of pulses, decreases compare to previous calculated rate of change values of impedance for previously emitted said pulses the controller is configured, to emit electrical energy via at least one electrode to ablate tissue at the determined voltage, for subjecting to an adipose layer or for subjecting to the adjacent tissue. The same apparatus may be used for determining an effective but safe voltage to use for ablation of the subjected tissue and for the actual ablation. This gives real time determination of the suitable voltage for ablation for that subjected tissue, whereas any extension time delay may actually mean that there may be a change in an optimal suitable voltage for ablation of the same tissue.

In specific embodiments, the earlier emitted pulse comprises the immediately prior emitted pulse.

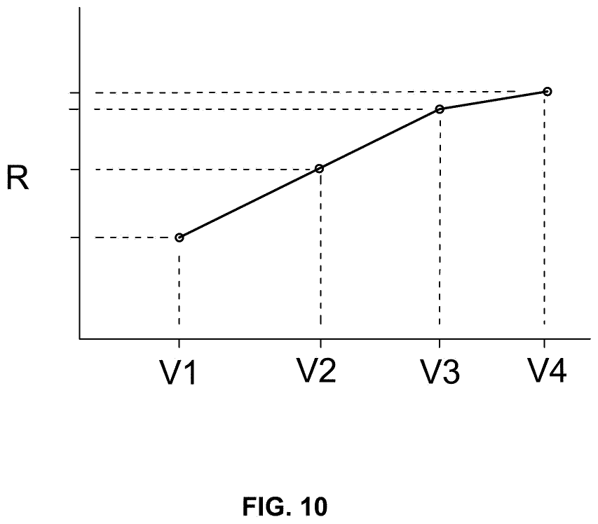

In some embodiments, the processor is further configured for, or comprises a configuration, to signal the controller that the voltage for ablation, is the voltage V of the earlier pulse, for example, the pulse N minus 1, where N was the number of the last pulse of the consecutive pulses, when the last determined impedance value of the last pulse emitted, has decreased in impedance value, or the rate of change of impedance in relation to the voltage has decreased. The rate of change of impedance in relation to voltage can be seen as the slope or curve of a graph, or the gradient of the graph, of voltage versus impedance when plotted on a graph. For example as shown in FIG. 10.

In some embodiments, the determined voltage for subjecting to an adipose layer and/or ablation of the target tissue within an adipose layer, for a pulse, or plurality of pulses of electrical energy is, the voltage value of the previous pulse emitted immediately before the pulse of increased voltage that had a decreased impedance compared to the impedance value of the prior, or earlier, or immediately before emitted pulse of electrical energy from a plurality of consecutive pulses comprising increasing voltage.

In some embodiments, the determined voltage for subjecting to an adipose layer and/or ablation of the target tissue within an adipose layer, for a pulse, or plurality of pulses of electrical energy is, the voltage value of the previous pulse emitted immediately before the pulse of increased voltage that had a decreased rate of impedance in relation to voltage compared to the rate of impedance value, in relation to voltage of the prior, or earlier, or immediately before emitted pulse of electrical energy from a plurality of consecutive pulses comprising increasing voltage.

In some embodiments, the controller is further configured for, or comprises a configuration, that when signalled that the impedance Z has decreased in value, or is less than, for a pulse from the plurality of pulses comprising increasing voltage, compared to the impedance value with previous pulse, the controller is configured to stop increasing the voltage of the pulses of electrical energy to be emitted. This reduces the risk that further pulses or electrical energy, is emitted of a high voltage that may harm non-adipose tissue.

In some embodiments, the controller is further configured for, or comprises a configuration, that when signalled that the impedance Z has decreased in value, or is less than, for a pulse from the plurality of pulses comprising increasing voltage, compared to the impedance value of the previous emitted pulse, the controller is configured, or comprises a configuration, to indicate to a user that the apparatus is ready for ablation mode. In some embodiments, the controller is further configured for, or comprises a configuration, that when signalled that the rate of change of impedance, in relation to voltage, has decreased in value, or is less than, for a pulse from the plurality of pulses comprising increasing voltage, compared to the rate of change impedance value, in relation to voltage of the previous emitted pulse, the controller is configured, or comprises a configuration, to indicate to a user that the apparatus is ready for ablation mode. Advantageously this enables a user to know when the apparatus has determined a suitable voltage for subjecting to an adipose layer and/or for ablation of the target tissue within an adipose layer. In some embodiments, the actual determined voltage is displayed. In some embodiments, the apparatus comprises a display screen. In some embodiments, the controller is further configured, or comprises a configuration, to display the determined voltage. Alternatively, in some embodiments, the apparatus may signal, by a visual or audial signal, that the apparatus is ready for use for ablation, or an ablation mode of the apparatus. In some embodiments, the controller is further configured, or comprises a configuration, to start automatically in the ablation mode of working after the processor has determined the voltage to use for subjecting to an adipose layer or for ablation of target cells within the adipose layer. In specific embodiments, the controller may be configured for, or comprise a configuration, to have any one or more of the combination of features: a visual signal that the voltage for ablation has been determined; a display of the determined voltage; an audial signal that the voltage has been determined; manual switching for the user to start ablation; automatic setting of the determined voltage for ablation; and automatic starting of ablation. Different embodiments with different features as described above allow the user their own level of control, and information required, of the apparatus.

In specific embodiments, the controller is further configured for, or comprises a configuration, to ablate the target tissue within the adipose tissue at the determined voltage for subjecting to an adipose layer. The skilled person will understand that the actual ablation may require a plurality of electrical energy over time directed to the target cells within an adipose tissue. Advantageously, this has efficiencies of time and material. The same device can be used to: 1, quantify the tissue to determine a voltage for subjecting to an adipose layer and wherein the voltage may be efficient for ablation of the target tissue within the adipose layer, while balancing less risk of comprising a voltage so high that nearby non-targeted, for example, non-adipose, tissue may be damaged; and 2, ablate the target tissue and in particular, for example, epicardial ganglionated plexi cells (GP). As the apparatus of the present invention can determine the voltage for subjecting to an adipose layer very quickly, for example, less than one second, the apparatus can be used for ablation practically straight away, the user may not notice any delay. In some embodiments, the processor may require a user to manually start ablation mode of the apparatus. As a check that the voltage is suitable or a set delay until the user is ready. Alternatively in some embodiments the apparatus will set the voltage for ablation to the determined voltage for ablation and proceed emitting pulses of electrical energy at that voltage when on.

In some embodiments, the controller is further configured for, or comprises a configuration, to emit pulses of electrical energy of the voltage for subjecting to an adipose layer or ablation of a target tissue within the adipose layer, or both. In specific embodiments, the voltage for subjecting to an adipose layer is the voltage as determined by the processor. Advantageously the electrodes used for the determining of the suitable voltage for subjecting to an adipose layer can also be used for the ablation. This enables only one apparatus to be used for both functions. This may enable a smaller apparatus to be used than in the prior art. There are cost, time and other advantageous efficiencies too that the same electrodes can be used for both the determining of the voltage suitable for subjecting to an adipose layer and for the actual ablation, for example of target cells within the adipose layer.

In specific embodiments, the controller is further configured for, or comprises a configuration, to emit pulses of electrical energy at the determined voltage for subjecting to an adipose layer, for example, of the adjacent adipose tissue, once the voltage for subjecting to an adipose layer is determined. The advantage of the present invention is that by measuring a relative value of the impedance of a portion of an adjacent tissue, for example, tissue that comprises adipose tissue, the apparatus may calculate a suitable voltage of an electrical energy pulse for subjecting that tissue to electrical energy and ablation of target cells within that tissue, for example adipose tissue. The apparatus may give, practically, real time appropriate information to specific tissue, for example tissue comprising adipose tissue. Advantageously this may mean that the apparatus of the present invention maybe able to quickly give a suitable voltage to ablation to a specific adipose tissue, by the relative value for the impedance of the tissue, or by the change, for example decrease, in the rate of change of impedance in relation to voltage/the change in slope or gradient, of impedance versus voltage on a graph of the values of impedance and voltage.

In some embodiments, the processor is further configured for, or comprises a configuration, to determine the impedance value for each pulse by using Ohm's Law. Ohm's Law, or Ohm's Law equation is Voltage equals current (I) times Impedance (Z) (V=I×Z). If a user, or the processor of the present invention, knows the voltage V and measure the current I then the impedance/Impedance Z can be calculated: Impedance (Z) equals voltage V over (or divided by) current (I); Z=V/I. As the invention uses relative values, and the electrical pulses are short in duration, calculating the resistance instead of impedance works too for the purposes of the invention and therefore the terms “Impedance” Z and Resistance “R” as used herein are used to mean the same, are equivalents and interchangeable terms. R=V/I is applicable.

The electrical energy source may be any pulsed electrical energy source able to convey energy to the cells. Typically, the ultimate electrical energy source is mains electricity or a generator producing pulsed electricity. In some embodiments, the energy source may comprise a battery. In some embodiments, the electrical energy source comprises an output pulsed voltage in the range of 10 to 3001 Volts In some embodiments, the electrical energy source comprises an output pulsed voltage in the range of 20 to 3001 Volts In some embodiments, the electrical energy source comprises an output pulsed voltage in the range of 49 to 3001 Volts. In some embodiments, the pulsed electrical energy comprises pulses between 45 and 3500 volts. In specific embodiments, the pulsed electrical energy comprises pulses between 50 and 3000 volts. In some embodiments, the electrical energy source comprises an output pulsed voltage in the range of 45 to 1500 Volts The range of voltage for the pulse of electrical energy may enable a range of adipose tissues, by type and thickness for example, to be measured for a suitable voltage for ablation and indeed consequently for ablation of the tissue. Preferably efficient ablation of the tissue, ablating all or most of the tissue, at a voltage value likely to be less risk of damaging untargeted tissue, for example heart muscle tissue.

In some embodiments, the energy source comprises Direct Current (DC). In alternative embodiments, the energy source comprises Alternative Current (AC).

In some embodiments, the interval between the emitted pulsed electrical energy is 1 second. In some embodiments when the apparatus is in testing, or measuring mode, when the apparatus is determining the voltage for subjecting to an adipose layer, the time interval between pulses may be shorter than when the apparatus is in ablation mode. Likewise, when the apparatus is in testing or measuring mode, when the apparatus is determining the voltage for subjecting to an adipose layer, the pulse duration may be shorter than the pulse duration when the apparatus is in ablation mode. In some embodiments, the processor is further configured for, or comprises a configuration, that the duration of the pulses for determining the voltage for ablation of an adipose tissue is less than the duration of pulses of electrical energy during the ablation mode. In some embodiments, the processor is further configured for, or comprises a configuration, that the frequency of the pulses for determining the voltage for subjecting to an adipose layer is greater than the frequency of pulses of electrical energy during the ablation mode.

In some embodiments, the processor is further configured for, or comprises a configuration, that in determining the voltage for subjecting to an adipose layer, the total number of pulses emitted for determining the voltage will be less than 200, or 150, or 100, or 70 pulses. This may enable the determination of the voltage for subjecting to an adipose layer, to be determined, before the testing pulses can do any harm to the tissue, as are low in number.

In specific embodiments, the controller is further configured for, or comprises a configuration, to via the said electrode, ablate epicardial ganglionated plexi (GP) cells, for example, within an adipose layer. Although the adipose layer is subjected to the electrical energy and may be described as ablated, or subject to the electrical energy for ablation, the target cells are not necessarily adipose cells but cells within the adipose tissue, for example, epicardial ganglionated plexi (GP) cells. Due to different susceptibility of the cells to the electrical energy the actual adipose tissue within the adipose layer may be practically unharmed, or less harmed, than the target cells, for example, the ganglionated plexi cells (GP).

In some embodiments, the apparatus further comprises a catheter. A catheter may house the electrodes. A catheter may also enable, or assist with, insertion and positioning of the electrodes within the patient. Similarly, the catheter may assist in the distribution of any saline solution required to be used.

In some embodiments, the apparatus further comprises an aspiration, or suction, pump. The aspiration or suction pump may assist in the removal of fluids, for example excess fluids between the electrodes and the adjacent tissue so to remove excess fluid that may influence impedance measurements.

In another aspect of the present invention there is provided an apparatus for ablation of an adipose tissue comprising an apparatus for determining a voltage of an electrical energy for subjecting to an adipose layer, for example, as herein described.

In specific embodiments the controller is configured to enable emitting a pulse of electrical energy, of a known voltage, from an at least first electrode positioned in electrical communication via an adjacent tissue with an at least second electrode and measuring the impedance of the said pulse of electrical energy, then further configured to enable
emitting a further pulse of electrical energy of known voltage, wherein the value of the voltage is greater than the value of the voltage of the previous said pulse and measuring the impedance value and determining if there is a change, for example decrease, in the impedance value, if not a change, or decrease in value, then repeating again emitting a further pulse with an increased value of known voltage, until a change in impedance is detected. The voltage value of the immediately prior emitted said pulse to the said pulse that had a decreased impedance indicates a suitable voltage for ablation of that tissue. Once a suitable value of voltage has been determined no further pulses are required for determining the suitable value, although this voltage may be maintained for ablation purposes.

In specific embodiments the controller is configured to enable emitting a pulse of electrical energy, of a known voltage, from an at least first electrode positioned in electrical communication via an adjacent tissue with an at least second electrode and measuring the impedance of the said pulse of electrical energy, then further configured to enable
emitting a further pulse of electrical energy of known voltage, wherein the value of the voltage is greater than the value of the voltage of the previous said pulse and measuring the rate of change of the impedance value, in relation to the voltage, between 2 or more consecutive said pulses and determining if there is a change, for example decrease, in the rate of change of impedance value in relation to the voltage of said pulses, if not a change, or decrease in value, then repeating again emitting a further pulse with an increased value of known voltage, until a change, for example decrease, in the rate of change of impedance is detected. The voltage value of the immediately prior emitted said pulse to the said pulse that had a decreased impedance indicates a suitable voltage for ablation of that tissue. Once a suitable value of voltage has been determined no further pulses are required for determining the suitable value, although this voltage may be maintained for ablation purposes.

A person skilled in the art would understand that this may work the other way around in that the apparatus of the invention could be configured to emit pulses of a high voltage initially and the consecutive pulses decrease in voltage until the impedance changes by increasing in value or increasing in value, for example by 10 percent, or 15 percent or 20 percent, or other percentage, or the rate of change of impedance in relation to the voltage increases, for example by 10 percent, or 15 percent or 20 percent, or other percentage.

In another aspect of the present invention there is provided a system for determining a voltage of electrical energy for subjecting to an adipose layer; or for ablation target cells within an adipose layer; or both, for the determining of a voltage for subjecting to an adipose layer and for ablation of the target tissue within an adipose layer, comprising an apparatus for determining a voltage for subjecting to an adipose layer, for example, as herein described.

In some embodiments the system for determining a voltage for subjecting to an adipose layer, further comprises an aspiration, or suction, pump. The aspiration or suction pump may assist in the removal of fluids, for example excess fluids between the electrodes and the adjacent tissue so to remove excess fluid that may influence impedance measurements.

In some embodiments of the system, the system further comprises any one or more from the following group: a catheter configured to house the electrodes; an energy source; a saline source; a fluid aspiration/suction source; and a display screen configured to display the determined voltage for ablation.

In a further aspect of the invention there is provided a method of determining a voltage for subjecting to an adipose layer, comprising the steps of:
positioning at least, a first electrode and second electrode, adjacent a tissue comprising adipose tissue, wherein the said electrodes are spaced apart and are of different polarity;
emitting, via at least one of the electrodes, or the first electrode, a pulse of electrical energy of a known voltage, and,
measuring the electrical current of the pulse of electrical energy;
calculating, using the known voltage of the emitted electrical energy and the measured current through a portion of the adjacent tissue, an impedance value of the adjacent tissue;
determining a voltage for the ablation of the adipose layer by comparing the calculated impedance value of the adjacent tissue, with one or more previously calculated impedance values.

In some embodiments wherein the adipose layer for subjecting electrical energy to, comprises epicardial Ganglionated Plexi cells (GP). In some embodiments the method comprises the step of providing the adipose layer for subjecting electrical energy wherein the adipose tissue comprises epicardial Ganglionated Plexi cells (GP).

In some embodiments the method comprises the step of positioning both an at least first and an at least second electrodes adjacent a tissue. In some embodiments the method comprises the step of positioning both an at least first and an at least second electrodes adjacent a tissue, wherein both the at least first and the at least second electrodes are in contact with the an adjacent tissue such that the electrodes are in electrical communication with each other vi the said adjacent tissue. This may enable the electrical circuit between a first electrode and a second electrode through the adjacent tissue to be completed.

In some embodiments, of the method of determining a voltage for subjecting to an adipose tissue, there comprises the further step of:
using ohms law equation to calculate an impedance value of the adjacent tissue.

Advantageously, the method and device of the present invention may determine a voltage for subjecting to an adipose layer, that the determined voltage is great enough to ablate at least some of the target tissue within the adipose layer subject to the electrical energy.

In some embodiments the method and or device of the present invention may determine a voltage for subjecting to an adipose layer, that the determined voltage is great enough to ablate at least some of the target tissue within the adipose layer subject to the electrical energy.

In some embodiments, the method, and or device of the present invention may be configured to determine a voltage for subjecting to an adjacent tissue layer, for example comprising adipose tissue, that the determined voltage is great enough to create an electrical field that encompasses at least 50 percent of the thickness of the said adjacent tissue without encompassing underlying muscle tissue to the said adjacent adipose tissue layer.

In some embodiments, the method, and or device of the present invention may be configured to determine a voltage for subjecting to an adjacent tissue layer, for example comprising adipose tissue, that the determined voltage is great enough to create an electrical field that encompasses at least 75 percent of the thickness of the said adjacent tissue without encompassing underlying muscle tissue to the said adjacent adipose tissue layer.

In some embodiments, the method, and or device of the present invention may be configured to determine a voltage for subjecting to an adjacent tissue layer, for example comprising adipose tissue, that the determined voltage is great enough to create an electrical field that encompasses at least 80, or 85, or 90, or 95, or 99 percent of the thickness of the said adjacent tissue without encompassing underlying muscle tissue to the said adjacent adipose tissue layer.

In some embodiments of the method of determining a voltage for subjecting to an adipose layer, there comprises the further step of:
determining a voltage for subjecting to an adipose layer by comparing the calculated impedance value of the adjacent tissue, comprising adipose tissue, with one or more previously calculated impedance values, wherein the adipose tissue comprises a thickness and the pulse of determined voltage extends through a portion of the thickness of the adipose tissue in the range of between 75 and 99 percent, or 50 to 99 percent, or 50 to 90 percent, or 75 to 90 percent, of the thickness of the adipose tissue, perpendicularly from the electrode.

For convenience of explaining the invention, the electrical energy has been, for some embodiments been described with increasing voltage of pulses, of electrical energy. Alternatively, in other embodiments the voltage of the pulses may decrease over time, or vary over time of the emission of the electrical energy.

In some embodiments of the method of determining a voltage for subjecting to an adipose layer, the electrical current of the emitted electrical energy extends through, or beyond, at least a portion, of the adipose tissue of the adjacent tissue, wherein the adjacent tissue comprises adipose tissue.

In some embodiments of the method of determining a voltage for subjecting to an adipose layer, there further comprises the step of:
repeating the emitting of a pulse of electrical energy wherein the consecutive pulse comprises a greater voltage and determining the impedance value for the consecutive pulses of electrical energy by measuring the electrical current of each of the pulses of electrical energy.

Having a plurality of pulses wherein each consecutive pulse comprising an increasing voltage value enables the processor to compare at least two pulses of different voltage.

In some embodiments of the method of determining a voltage for subjecting to an adipose layer, further comprises the step of:
repeating the emitting of a pulse of electrical energy wherein the consecutive pulse has a greater voltage and determining the impedance value, and or the rate of change of impedance value in relation to voltage, for the consecutive pulses of electrical energy by measuring the electrical current of each of the pulses of electrical energy, until the impedance value of a pulse decreases, or the rate of change of the impedance decreases in relation to voltage, in comparison to the impedance value, or rate of change, of a previous pulse.

As adipose tissue has a greater impedance than some other tissue, for example muscle tissue, the apparatus of the invention is able to detect by the impedance measurements and comparing to the impedance measurements for different voltages if the electrical field or electrical energy of a pulse captures, or extends through only adipose tissue, for example, the adjacent tissue comprising adipose tissue; or extends beyond the adipose tissue of the adjacent tissue, and also captures other tissues, or tissue types, for example, underlying muscle tissue, and thus the muscle tissue is subject to electrical energy too. As the muscle tissue has a much less impedance value than an adipose tissue, when the electrical energy of the pulse extends beyond the adipose tissue to also capture some muscle tissue, the impedance value will be less than if the pulse only captured adipose tissue alone. Alternatively, or at least, the rate of change of the impedance in relation to voltage will decrease when the electrical field encompasses muscle tissue compared to only encompassing adipose tissue. When the values plotted on a graph of impedance versus voltage the slope, or gradient, will fall, or decrease showing that the rate of change of impedance will fall, when the voltage increases, when the electrical field encompasses muscle tissue in addition to adipose tissue when a plurality of consecutive electrical pulses increase in voltage.

In some embodiments, the previous pulse comprises the immediately emitted previous pulse of a plurality of consecutively emitted pulses.

In some embodiments of the method of determining a voltage for subjecting to an adipose layer, further comprises the step of:
determining the voltage for subjecting to an adipose layer such that the voltage for ablation is equal to the voltage of the pulse of electrical energy emitted before the pulse of electrical energy comprising an impedance that decreased in value, in comparison to the previous pulse impedance value.

In some embodiments of the method of determining a voltage for subjecting to an adipose layer, further comprises the step of:
determining the voltage for subjecting to an adipose tissue layer such that the voltage for ablation is equal to the voltage of the pulse of electrical energy emitted before the pulse of electrical energy comprising a rate of change of impedance in relation to voltage that decreases, in comparison to the previous pulse impedance value.

In some embodiments of the method of determining a voltage for subjecting to an adipose layer, further comprises the step of:
calculating the impedance of a portion of an adjacent tissue by using the Ohm's law equation, or a form of the Ohm's Law equation.

In some embodiments of the method of determining a voltage for subjecting to an adipose layer, further comprises the step of:
calculating the rate of change of impedance, in relation to voltage, of a portion of an adjacent tissue by using the Ohm's law equation, or a form of the Ohm's Law equation in relation to time.

This enables the processor to compare the impedance value of the adipose tissue, or a portion of the adipose tissue calculated using different voltages. In embodiments with consecutive pulses of an increasing voltage and measuring the impedance values, the initial low voltage pulses of electricity may initially only capture, or be transmitted through, adipose tissue of the adjacent tissue, but as the voltage increases the electric field increases and there will be a point when the electric energy also captures muscle tissue, for example cardiac muscle tissue, too, as the electrical energy extends beyond the adipose tissue layer of the adjacent tissue. At this point, where the voltage captures some muscle tissue the impedance value will be less than the impedance value of the previous pulse, or not increase by as much, thus the rate of change of impedance value will be less than previously, when voltage of consecutive pulses increase, or when the pulse only captures the adipose tissue. The change, for example decrease, in the rate of change of impedance value in relation to voltage may be seen on a graph of these values, impedance versus voltage, that the curve will decrease or drop, this may indicate that the interface between the adipose tissue and muscle tissue has been reached by the pulse of electrical energy, as muscle has less impedance than adipose tissue.

In some embodiments of the method of determining a voltage for subjecting to an adipose layer, further comprises the step of:
displaying the voltage for subjecting to an adipose layer; or,
ablation of the target tissue within the adipose layer; or,
both, displaying the voltage for subjecting to an adipose layer and ablation of the target tissue within the adipose layer.

In some embodiments of the method of determining a voltage for subjecting to an adipose layer, there further comprises any one or more of the steps from the group:
subjecting the adipose layer to pulses of electrical energy of the determined voltage for subjecting to the adipose layer; or,
subjecting the target cells within the adipose layer to pulses of electrical energy of the determined voltage for subjecting to the adipose layer; or,
ablating the target cells within the adipose layer at the determined voltage for subjecting to an adipose layer; and,
ablating some of the target cells within the adipose layer at the determined voltage for subjecting to an adipose layer.

In some embodiments of the method of determining a voltage for subjecting to an adipose layer, further comprises the step of:
ablating epicardial ganglionated plexi (GP) cells within the adipose tissue.

A person skilled in the art would understand that the terms as used herein: ablation of adipose tissue, or subjecting the adipose tissue to the ablation treatment, mean that the actual target cells for ablation may be particular cells, for example, epicardial ganglionated plexi cells (GP) within the adipose tissue. Often the target cells are more vulnerable, or sensitive, to the ablation treatment than other cells or tissue for example adipose tissue. The person skilled in the art would understand that the terms as used herein, of, “ablation of adipose layer or tissue” or “subjecting ablation electrical energy to an adipose layer or tissue” does not necessarily include killing the actual adipose tissue, that there may be target cells within the adipose layer or tissue, that may be more susceptible to the electrical energy.

In some embodiments of the method of determining a voltage for subjecting to an adipose tissue, there further comprises the step of:
actuating one or more of the, at least first electrode, or the at least second electrode, to emit electrical energy of a known voltage. Alternative embodiments may comprise any number of electrodes. In some embodiments, a plurality of electrodes are independently controlled or actuated. In alternative embodiments, the electrodes may be paired or grouped such that the pair, or group, of electrodes are actuated, or controlled together.

In some embodiments of the method of determining a voltage for subjecting to an adipose layer, further comprises the step of:
emitting pulses comprising different voltage.

This enables the processor to compare the impedance value of the adjacent tissue for example wherein the adjacent tissue comprises adipose tissue, calculated for pulses of different voltage, this includes comparing relative values of the impedance or impedance value.

In some embodiments of the method of determining a voltage for subjecting to an adipose layer, further comprises the step of:
calculating the impedance value of the plurality of pulses of different voltage.

In some embodiments of the method of determining a voltage for subjecting to an adipose layer, further comprises the step of:
calculating the rate of change of impedance in relation to voltage of the plurality of pulses of different voltage.

In some embodiments of the method of determining a voltage for subjecting to an adipose layer, further comprises the step of:
displaying the voltage value of the voltage determined for subjecting to an adipose layer; or
displaying the voltage value of the voltage for ablation of target tissue within an adipose layer; or
both, displaying the voltage value of the voltage determined for subjecting to an adipose layer and displaying the voltage value of the voltage for ablation of target tissue within an adipose layer.

In some embodiments of the method of determining a voltage for subjecting to an adipose layer, further comprises the step of:
displaying the impedance value of the portion of adjacent tissue, for example wherein the adjacent tissue comprises adipose tissue.

Some embodiments comprise the further step of:
displaying a graph of impedance versus voltage and using the change of slope to indicate the suitable voltage for ablation or the interface of tissue between adipose tissue and muscle tissue.

In some embodiments of the method of determining a voltage for ablation of an adipose tissue, further comprises the step of:
determining a voltage able to ablate, for example, epicardial ganglionated plexi cells, within the portion of adjacent tissue, by comparing to known values of voltage suitable for ablation of epicardial ganglionated plexi cells within known thicknesses of adipose tissue.

In some embodiments, of a method of determining a voltage for subjecting to an adipose layer, there is the further step of:
actuating at least a first electrode that at least a first pulse of electrical energy and at least a second pulse of electrical energy are of a different voltage value. The different voltage values of the pulses of electrical energy may result in different electrical field strength and may give a difference in impedance value that the processor is configured, or comprises a configuration, to determine. The different impedance values can reveal if the electrical field of the pulse captures just adipose tissue or captures both adipose tissue and another, for example, muscle tissue, thus the electrical energy extends beyond the adipose tissue of the adjacent tissue comprising adipose tissue.

In some embodiments, of a method of determining a voltage for subjecting to an adipose layer there is the further step of:
actuating at least a first electrode to emit at least a second pulse of electrical energy that comprises a greater voltage value than the at least first pulse of electrical energy.

In some embodiments, of a method of determining a voltage for subjecting to an adipose layer there is the further step of:
actuating at least a first electrode to emit a number of pulses of electrical energy wherein the number of pulses is n; and,
each pulse has a known voltage V. In some embodiments, the specific voltage V to the specific pulse n is Vn.

In some embodiments, of a method of determining a voltage for subjecting to an adipose layer there is the further step of:
increasing the voltage V for each consecutive pulse increasing in number n and measuring the electrical current of each pulse n.

In some embodiments, of a method of determining a voltage for subjecting to an adipose layer, there is the further step of:
conveying information that the current sensor measures to the processor. This enables the processor to use the information.

In some embodiments, the electrical components, for example, the controller, the electrodes and the processor are in electrical communication with each other. The person skilled in the art would understand this to be the case, that the electrical components can communicate and transfer data between each other as needed.

In some embodiments, of a method of determining a voltage for subjecting to an adipose layer there is the further step of:
calculating an impedance value Z, from the known voltage V and the electrical current I for each pulse n, and monitoring the impedance values, comparing the impedance value Z for each pulse n with the impedance value of the earlier emitted pulse, including at least the immediately emitted. The immediately emitted pulse may be described as N minus 1, where N is the greatest number of consecutive pulses emitted.

In some embodiments, of a method of determining a voltage for subjecting to an adipose layer there is the further step of:
signalling to the controller upon detection that, the impedance value Z of the pulse n, decreases, or is less than, in value compared to the impedance value Z of the earlier emitted pulse. In some embodiments, the earlier pulse comprises the immediately earlier pulse, N minus 1, of a plurality of pulses where N is the greatest number of pulses in a plurality of consecutive pulses comprising increasing voltage values.

In some embodiments, of a method of determining a voltage for subjecting to an adipose layer there is the further step of:
signalling to the controller upon detection that, the rate of change of impedance value Z of the pulses n in relation to voltage, decreases, or is less than, in value compared to the rate of change of impedance value Z in relation to voltage of the earlier emitted pulse. In some embodiments, the earlier pulse comprises the immediately earlier pulse, N minus 1, of a plurality of pulses where N is the greatest number of pulses in a plurality of consecutive pulses comprising increasing voltage values.

In some embodiments, of a method of determining a voltage for subjecting to an adipose layer there is the further step of:
signalling the controller that the voltage for ablation, is the voltage V of the earlier pulse, for example the pulse N minus 1, wherein the pulse with the greatest value of N, or Voltage, had an impedance Z that was less than the impedance Z of the pulse emitted before, for example emitted immediately before, N minus 1, of a plurality of consecutive pulses comprising an increasing voltage.

In some embodiments, of a method of determining a voltage for subjecting to an adipose layer there is the further step of:
signalling the controller that the voltage for ablation, is the voltage V of the earlier pulse, for example the pulse N minus 1, wherein the pulse with the greater voltage value had decreased rate of change of impedance in relation to voltage, than the rate of change of impedance of the pulse emitted before, for example emitted immediately before, of a plurality of consecutive pulses comprising an increasing voltage.

In specific embodiments of the method of determining a voltage for subjecting to an adipose layer, there is the further step of increasing, or decreasing, the voltage value of the pulse in steps of any one or more of the following groups: 0.1 volts, 0.5 volts, 1 volt; 2 volts; 5 volts; 10 volts; 20 volts; 50 volts; 100 volts and 1000 volts.

In some embodiments, of a method of determining a voltage for subjecting to an adipose layer there is the further step of:
stopping emission of pulses comprising an increased voltage when the voltage for ablation of the adipose tissue is determined; or
preventing an increase in the voltage of the pulses emitted, greater than the determined voltage for ablation. This could be until the apparatus is reset, or repositioned.

In specific embodiments the method of determining a voltage for subjecting to a tissue, for example adipose tissue comprises the step of emitting a pulse of electrical energy, of a known voltage, from an at least first electrode positioned in electrical communication via an adjacent tissue with an at least second electrode and measuring the impedance of the said pulse of electrical energy, then
emitting a pulse of electrical energy of known voltage, wherein the value of the voltage is greater than the value of the voltage of the previous said pulse and measuring the impedance value and determining if there is a change, for example decrease, in the impedance value, if not a change, or decrease in impedance value, then repeating again emitting a further pulse with an increased value of known voltage, until a change in impedance is detected, giving a suitable voltage for ablation of that tissue. Once a suitable value of voltage has been determined no further pulses are required for determining the suitable value, although this voltage may be maintained for ablation purposes.

In specific embodiments the method of determining a voltage for subjecting to a tissue, for example adipose tissue comprises the step of: emitting at least two pulses of electrical energy, of a known increasing voltage, from an at least first electrode positioned in electrical communication via an adjacent tissue with an at least second electrode and measuring the impedance of the said pulse of electrical energy, then
emitting another pulse of electrical energy of known voltage, wherein the value of the voltage is greater than the value of the voltage of the previous said pulse and measuring the impedance value and determining if there is a change, for example decrease, in the rate of change of impedance in relation to voltage, if not a change, or decrease in impedance value, then repeating again emitting a further pulse with an increased value of known voltage, until a rate of change in impedance in relation to voltage is detected, giving a suitable voltage for ablation of that tissue, that is the voltage value of the immediately previous emitted said pulse. Once a suitable value of voltage has been determined no further pulses are required for determining the suitable value, although this voltage may be maintained for ablation purposes.

In specific embodiments the method of determining a voltage for subjecting to a tissue comprises the step of: emitting at least two, or at least three, pulses of electrical energy of different known voltage. In specific embodiments the subsequent emitted pulse comprises an increased voltage value over the previous emitted voltage.

In specific embodiments the method of determining a voltage for subjecting to a tissue, for example adipose tissue comprises the step of: emitting at least two pulses of electrical energy, of a known different increasing voltage, from an at least first electrode positioned in electrical communication via an adjacent tissue with an at least second electrode and measuring the impedance of the said pulse of electrical energy, then
emitting another pulse of electrical energy of known voltage, wherein the value of the voltage is greater than the value of the voltage of the previous said pulse and measuring the impedance value and determining if there is a change, for example decrease, in the rate of change of impedance in relation to voltage, if not a change, or decrease in the rate of change of impedance in relation to voltage, then repeating again emitting a further pulse with an increased value of known voltage, until a rate of change in impedance in relation to voltage is detected for example decreases, giving a suitable voltage for ablation of that tissue, that is the voltage value of the immediately previous emitted said pulse with the higher rate of impedance. Once a suitable value of voltage has been determined no further pulses are required for determining the suitable value, although this voltage may be maintained for ablation purposes.

In some embodiments, of a method of determining a voltage for subjecting to an adipose layer there is the further step of:
indicating to a user that the apparatus is ready for ablation, when the voltage for subjecting to an adipose layer has been determined. Advantageously this enables a user to know when the apparatus has determined a suitable voltage for ablation of the adipose tissue and the apparatus is ready for use for ablation.

In some embodiments, of a method of determining a voltage for subjecting to an adipose layer there is the further step of:
subjecting the adipose tissue to electrical energy, or ablating, at least some of the target cells, for example, epicardial ganglionated plexi (GP) cells, within the adipose tissue. This may be, in some embodiments, automatically performed, or that the device switches automatically to an ablation mode, once the voltage for ablation is determined, without any further input from the user.

In some embodiments, of a method of determining a voltage for ablation of an adipose tissue there is the further step of:
subjecting the adipose tissue to electrical energy of the determined voltage, or ablating, at least some of, the target cells, for example, epicardial ganglionated plexi (GP) cells, within an adipose tissue, for example, using the same electrode or electrodes used for determining the voltage for ablation.

In some embodiments, the adipose tissue comprises epicardial cells. In some embodiments, the target cells for ablation are epicardial ganglionated plexi (GP) cells.

The advantage of the method of the present invention is that by measuring a relative value of the impedance, or rate of change of impedance relative to the voltage, of an adjacent tissue, for example adjacent adipose tissue, the apparatus can calculate a suitable voltage of an electrical energy pulse for ablation of target cells within that adipose tissue. The method may be able to give, practically, real time information to specific adipose tissue. Advantageously this means that the method of the present invention is quick to give a suitable voltage for ablation to cells within a specific adipose tissue, by relative value for the impedance of the tissue.

Although for the sake of clarity to describe the invention, and in preferred specific embodiments, the consecutive pulses have an increased voltage; it is the difference in the impedance calculated from the measured electrical current that is a significant event and feature of the invention. Or the different in the rate of change of the impedance in relation to voltage that is significant. For increasing voltage of the pulses a significant event is when the impedance, or rate of change of impedance in relation to voltage, decreases, indicating that the electric field is likely to have penetrated through the adipose tissue and reach adjacent muscle tissue. This however can work the other way round too, that a change in the calculated impedance value, for example increasing, coming from the other direction of a plurality of consecutive pulses comprising decreasing voltage. Likewise for calculating the rate of impedance in relation to voltage.

In some embodiments, the voltage may start high and consecutive pulses have a decreasing voltage and when the impedance increases this may indicate that the voltage used to give this increased impedance compared to the impedance value of the immediately previous pulse, as a relative value, is the determined voltage to use for subjecting to the adipose tissue and or for ablation of target cells within the adipose tissue.

Likewise, in some embodiments, the voltage of the consecutive pulses may be alternated, or be random, of high and low voltages, honing in to the voltage for ablation, wherein, the determined voltage for subjecting to an adipose layer and/or ablation of the target cells within the adipose tissue is:
the voltage value of the pulse n that is less than the voltage value of a pulse that has a decrease change in impedance compared to the impedance value of a lower voltage pulse; or,
the voltage value of the pulse n that is greater than the voltage pulse that has an increase change in impedance compared to the impedance value of a higher voltage pulse. This is for the incremental order of the pulses with increasing or decreasing voltage that the change of impedance value compared to the general trend of increasing or decreasing is the signal that:
a/ the electrical field has extended into non-adipose tissue, in the case of the impedance value of a consecutive voltage increasing pulse decreases in impedance value; or,
b/ the electrical field is extended only into adipose tissue, where before it was also extended into non-adipose tissue for example muscle, in the case of the impedance value of a consecutive voltage decreasing pulse increases in impedance value.

In further embodiments, the processor is configured to choose voltage values of a pulse of electrical energy based on the information obtained from previous emitted pulses. In such embodiments, that the processor may choose particular voltages for the pulses emitted, the processor is configured to include both, pulses of increasing voltage value and decreasing voltage value. The processor may therefore quickly fine tune to determine the voltage for ablation. In some alternative embodiments the pulse duration may be in the nanosecond range, 1 or 2 nanoseconds, or between 1 and 2 nanoseconds.

In some embodiments, the duration of the pulsed electrical energy is between 5 and 250 microseconds μs. In specific embodiments, the duration of the pulsed electrical energy is between 10 and 200 microseconds μs.

In specific embodiments, the pulse of the electric field comprises a pulse duration of 100 microseconds μs. In some embodiments, the pulse of the electric field comprises a pulse duration between 30 and 200 microseconds μs. In some embodiments, the pulse of the electric field comprises a pulse duration between 90 and 110 microseconds μs.

The duration of the pulses can be the same, or different for the determining of the voltage for ablation and for the actual ablation. In specific embodiments, the duration of the pulse of electrical energy emitted for determining the voltage for subjecting to an adipose layer is less than the duration of pulses of electrical energy used for ablation. In specific embodiments, the duration of pulses of electrical energy for the determining of the voltage for ablation is less than 100 or 60 or 50 microseconds μs.

In some embodiments, the pulse frequency is in the range of 30 to 200 pulses per minute (BPM). In some embodiments, the pulse frequency is in the range of 60 to 120 pulses per minute (BPM). In some embodiments, the pulse delivery is in the range of 0.3 to 3 Hertz (Hz). In alternative embodiments, the pulse frequency is in the range of 0.5 to 1 Hertz (Hz). In specific embodiments, the pulsed delivery is synchronised with the Ventricular Refractory period of the heart of the patient. The Ventricular Refractory period of the heart of a patient can be easily determined by means known to those skilled in the art. In some embodiments, the pulse interval is in the range of 0.1 to 1 seconds. The pulse interval is the space between one pulse ending and the next pulse starting. In some embodiments, the interval between the emitted pulsed electrical energy is 1 second.

In some embodiments, the apparatus comprises a saline source. The use of saline may assist in the electrical contact with the adipose tissue, for determining the voltage for ablation.

In some embodiments, the catheter comprises insulation material. In some embodiments, the catheter comprises an insulation layer. In specific embodiments, the catheter comprises an insulation layer at, or towards, the back of the catheter. The back of the catheter is the non-targeted direction, or portion not comprising an opening, or portion opposite the opening. In some embodiments, the catheter comprises an insulation layer surrounding the openings of the catheter. In some embodiments, the wall of the catheter comprise an insulating layer.

In some embodiments, the insulation material comprises a polymer. In some embodiments, the insulation material comprises PEBAX, a Trade Name for a polymer.

The catheter is typically configured to aid positioning within the body. Typically, the catheter is elongated in shape; and pointed, rounded or smooth, at one end, for example the distal end. In some embodiments, one end, for example, the distal end of the catheter comprises a tapered, or pointed, or acuminate shape. In some embodiments, the catheter comprises an atraumatic shaped end. This helps to enable positioning of the catheter within the patient and to reach a good position for any ablation procedure. Ideally, for epicardial ablations the catheter is configured to be able to be inserted into the pericardial space at an entry point. In some embodiments, the distal end of the catheter is configured for insertion into the pericardial space at an entry point.

The electrodes for the present invention can be any known electrode suitable for conveying energy within the body of the patient. Ideally, the, or at least one electrode is at least partially housed within the catheter. The electrodes may comprise platinum iridium or stainless steel for example.

In some embodiments, the catheter comprises a plurality of electrodes. In some embodiments, the catheter comprises a plurality of positive electrodes. In some embodiments, the catheter comprises a plurality of negative electrodes. In some embodiments, the catheter comprises a plurality of electrodes, both positive and negative electrodes. In some embodiments, the catheter comprises a row of alternating positive and negative electrodes.

In some embodiments, the catheter comprises a plurality of both cathode and anode electrodes, wherein the electrodes alternate in pairs of cathode and anode electrodes along the longitudinal length of the catheter.

In some embodiments the outer radially facing surface of the electrodes are between 2 and 4 millimetres in length and between 1.5 and 2.5 millimetres in width. In specific embodiments, the electrodes comprise an outer diameter (OD) of 2.2 mm, an inner diameter (ID) of 1.9 mm a length, longitudinal axis wise, of 3.18 mm. In alternative specific embodiments, the electrodes comprise an outer diameter (OD) of 3.759 mm, inner diameter (ID) of 3.429 mm, and a length of 3.18 mm. In some embodiments, the diameter of the catheter is approximately 8.5 French. In some specific embodiments the radially facing surface of the electrodes are 3.2 by 2.5 millimetres. In some alternative embodiments the radially facing surface of the electrodes are 2.7 by 2 millimetres.

In some embodiments, the catheter comprises an opening. In some embodiments the catheter comprises an opening, wherein the opening is positioned at, or near to, one end of the catheter, for example the distal end. In some embodiments the opening is configured to expose a portion of the at least one electrode, for example, to the outside of the catheter. In some embodiments the electrode is larger than the size of the exposed opening. In some embodiments, the opening of the catheter is half the distance of the circumference of the catheter across the longitudinal axis. In other embodiments, the opening is less than half the distance, or less than 180 degrees, of the circumference of the catheter across the longitudinal axis. In some embodiments, the opening is between a quarter and a half of the circumference (distance) of the catheter, or between 90 and 180 degrees, of the circumference of the catheter, across the longitudinal axis. In some embodiments, the opening is between an eighth and a half of the circumference (distance) of the catheter, or between 45 and 180 degrees, of the circumference of the catheter, across the longitudinal axis. In some embodiments, the opening is between an eighth and a quarter of the circumference (distance) of the catheter, or between 45 and 90 degrees of the circumference of the catheter, across the longitudinal axis. In some embodiments, the opening is between 0.5 and 5 millimetres in length across the longitudinal axis direction of the catheter. In specific embodiments, the opening is between 1 and 3 millimetres in length across the longitudinal axis direction of the catheter. In specific embodiments, the opening is 2 millimetres in length across the longitudinal axis direction of the catheter. In some embodiments, the opening is between 2 and 3 millimetres in length along the longitudinal axis direction of the catheter. In specific embodiments, the opening is between 2.5 and 2.9 millimetres in length along the longitudinal axis direction of the catheter. In specific embodiments, the opening is 2.7 millimetres in length along the longitudinal axis direction of the catheter. The opening allows directional targeting of the electric field. In some embodiments, the opening is adjustable in size. In some embodiments, the opening is adjustable in its facing position. The opening assists in enabling the electric field direction to be determined, and thus assist in targeting particular cells or tissue with the electric field, for ablation.

In some embodiments the outer radially facing surface of the electrodes are between 2 and 4 millimetres in length and between 1.5 and 2.5 millimetres in width. In specific embodiments, the electrodes comprise an outer diameter (OD) of 2.2 mm, an inner diameter (ID) of 1.9 mm a length, longitudinal axis wise, of 3.18 mm. In alternative specific embodiments, the electrodes comprise an outer diameter (OD) of 3.759 mm, inner diameter (ID) of 3.429 mm, and a length of 3.18 mm. In some embodiments, the diameter of the catheter is approximately 8.5 French. In some specific embodiments the radially facing surface of the electrodes are 3.2 by 2.5 millimetres. In some alternative embodiments the radially facing surface of the electrodes are 2.7 by 2 millimetres.

In some embodiments, the catheter comprises a shaft wall. The shaft wall may comprise of a polymer, for example PEBAX, a Trade Name for a polymer. The shaft wall of the catheter may comprise a window or opening. In some embodiments the shaft wall comprises braiding lined with polymer, for example polytetrafluoroethylene PTFE polymer, for radial and axial support. In specific embodiments, the catheter further comprises an insulating conduit to partially at least house electrical wiring. The frame or casement of the window or opening may, in some embodiments, be raised radially from the longitudinal axis of the catheter from the wall of the main shaft of the catheter. The frame or casement of the window or opening of the catheter may comprise a radially projecting distance from the longitudinal axis of the catheter that is between 0.1 and 1.2 millimetre greater than the radially projecting distance of the wall of the main shaft of the catheter from the longitudinal axis of the catheter. The frame of the window or opening of the catheter may comprise a radially projecting distance from the longitudinal axis of the catheter that is between 0.1 and 0.6 millimetre greater than the radially projecting distance of the wall of the main shaft of the catheter from the longitudinal axis of the catheter. Having the frame or casement of the window or opening raised from the wall of the catheter enables the electrode to sit proud of the wall of the shaft of the catheter and thus enables a better contact of the catheter with the tissue. As the tissue is soft, the electrode protruding from the catheter will push slightly into the tissue giving a good contact, a good electrical connecting contact, that the electric field will pass into the tissue. Advantageously the raised frame or casement of the window can aid in the prevention of concentrated energy at the edges of the electrodes.

In general, the device of the present invention will have a pair of oppositely charged electrodes positioned near to the heart of a patient, for example positioned adjacent to the epicardial fat pad of the patient. The electrodes will be in electrical contact, directly or indirectly with the adjacent tissue, for example a tissue comprising adipose tissue and as further example, an adipose tissue comprising ganglionated plexi cells. Often the electrodes will be housed, in a catheter comprising at least one pair of electrodes configured to be positioned near to, or adjacent to the adipose tissue for example cardiac adipose tissue around the heart of the patient. Techniques are known, for insertion of medical devices within the body, and these may or may not include positioning devices. Ideally, in some embodiments, the catheter may comprise both the anode and cathode electrodes and thus the electric field will mainly be between the electrodes of the catheter, and the near surrounding area, adjacent tissue for example, and more particular adjacent tissue comprising adipose tissue. In some methods of using the device, the pair of electrodes, one a cathode and one an anode, are positioned so as to target the electric field through the targeted tissue or cell bodies, for example the ganglionated plexi neuron cell bodies of the patient. When the at least first and second electrodes are in position in use and therefore in electrical contact directly or indirectly with an adjacent tissue, for example tissue comprising adipose tissue, the adjacent tissue completes the electrical flow and the current sensor can measure the current of a pulse of electrical energy.

In some use of the invention, typically, the distal end of the catheter will be inserted via an anterior entry point to the pericardial space of the heart of a patient. A portion of the catheter may contact the epicardial surface of the heart. In addition or alternatively, the exposed portion of the electrode may be positioned to face the epicardium of the heart of the patient. When in the desired position, the electrodes may be switched on and the electrode or electrodes activated, to produce the desired electric field, in the desired direction. Tissue, cells and cell bodies in the immediate area of the electric field will receive a higher energy level than tissue or cells further away from the electric field.

In a preferred embodiments of the present invention, the voltage of the pulse used to begin with, will start low, for example 50 Volts and the electrical current measured for each pulse of electrical energy. The processor will using Ohm's Law equation calculate an impedance value for the adjacent tissue and stores this value for the pulse of that particular known voltage. Typically, this process is repeated. Another pulse of electrical energy is emitted but with a slightly higher known voltage value, for example 55 volts, or 60 volts. The pulses are, or can be, extremely short in duration and intervals, and may be shorter in duration for the determining of the voltage for ablation mode, than actually used duration ablation mode, and thus the controller and processer can emit a large number of different pulse with different voltages, and record the current and calculate the impedance and indeed the voltage for ablation in a short space of time, for example, less than 1 second. The emitting of consecutive pulses of electrical energy, with known voltage is repeated and repeated as required, with a slightly increased voltage until there is a change in impedance detected. In the preferred embodiment wherein the consecutive voltages are increasing in voltage, the processor is configured, or comprises a configuration, to determine when the calculated impedance decreases in value compare to the impedance value of the previous pulse emitted. In some embodiments the change detected is a change in the rate of impedance in relation to the voltage and in particular a decrease in the rate of change of impedance in relation to voltage. As adipose tissue has a greater impedance to the electric field than muscle tissue this, decrease in impedance value, or at least decrease in the rate of impedance in relation to voltage, indicates that the electric field has penetrated though the adipose tissue and has reached and is passing through a portion of muscle tissue. Therefore the maximum voltage that has a reduce risk, unlikely to reach the muscle tissue is the voltage of the previous pulse emitted that had a greater impedance value, or greater rate of change of impedance in relation to voltage, calculated than the last voltage value of the last emitted pulse, where the impedance value, or rate of impedance, decreased compare to the previous pulse of electrical energy.

The person skilled in the art will understand that this can work the other way too, starting with a high voltage value but for safety it is advantageous to start low voltage to avoid the risk of penetrating through the adipose tissue and damaging muscle tissue or at least to lower the duration at high voltage of penetrating the adipose tissue and exposing the muscle tissue adjacent to electrical energy.

In addition, tissue or cells in the targeted direction of the electric field will receive a greater energy level than tissue or cells not in the targeted direction and area of the electric field. The device may be repositioned as desired to target other tissue or cells, but also to concentrate the energy on the desired targeted tissue and cells while reducing the energy or period of exposure of the energy of non-targeted tissue or cells. In addition, saline may be dispensed from the catheter, for example from the conduit of the catheter, through the port of the electrode to target the electric energy and electric field to the targeted area and cell bodies. The saline may assist in concentrating the electrical energy or electric field strength in the desired targeted area and to the desired targeted cell bodies, for example ganglionated plexi neuron cell bodies. On completion of the procedure, the catheter and other components of the device are removed from the patient and the insertion holes sealed.

Cells and tissue, for example, the ganglionated plexi neuron cell bodies, that receive a higher electric field strength or a longer exposure to an electric field strength, or a combination of higher field strength and longer duration of electric field, are more likely to be ablated or destroyed.

In a further aspect of the present invention there is provided the method of manufacturing a device as herein described.

In a further aspect of the present invention there is provided the use, of an apparatus or system as herein described, for the treatment, or ablation, of target cells for example, ganglionated plexi cells within an adipose layer or tissue.

### Terms

By the term “ablate” this is used to mean, killing or destroying a cell or tissue, or at least some cells or tissue, it need not be limited to total kill. The term includes ablation of target cells within a tissue, for example, ablation of a adipose layer or epicardial fat includes subjecting the adipose layer or epicardial fat tissue to a treatment, for example electrical energy, where the ablation is to target the epicardial ganglionated cells within the fat layer or epicardial fat tissue, and not necessarily kill or remove, all the cells within the tissue. Often the target cells are more susceptible to the treatment, thus not all cells within a tissue need to be removed or killed for effective ablation. The term may include in vitro application.

In an Alternating Current, commonly known “AC”, Impedance (Z) is the opposition to current flowing around the circuit. Impedance is a value given in Ohms or is measured in Ohms. In a Direct Current (DC), the opposition to current flow is called Resistance R but in an AC system, impedance is the result of both circuit resistive and reactive components. However, in the present invention as herein described, it is the relative values that are compared, and together with that, the electrical pulses are of a short duration, for example usually 50 microseconds or less, the terms “impedance” and “Impedance” are used herein to be equivalent, interchangeable, terms. Resistance R is also measured in Ohms. For the purposes of the invention, it does not matter if AC or DC is used for the electrical pulses.

By the term “adipose layer” as used, herein this term includes layers, of tissue and cells, that comprise adipose tissue but are not necessarily consisting completely of adipose tissue or cells, and includes layers where there are other types of cells or tissues, for example. The term includes in vitro tissue. The term “adipose layer” and “adipose tissue layer” are equivalents, in meaning as used herein.

By the term “adjacent tissue” or the like as used herein with reference to adjacent tissue or layer, this term includes directly adjacent to and indirectly adjacent to, for example, the electrode may be indirectly adjacent a tissue or layer of tissue via a salt solution. The term “adjacent” in reference to tissue, and the like; or “adjacent tissue” and the like, as used herein is to mean in electrical communication, directly or indirectly, with, for example, via a saline solution between the electrode and the tissue, but also includes the tissue in which the electrical energy from the electrode flows at least through a portion of. The term may also include through other tissues for example skin tissue. Where an at least first and second electrode is adjacent a tissue it means that the electrodes are in electrical communication with the said adjacent tissue.

By the term “captured” and the like in reference to tissue is used herein to mean tissue through which the electric field extends through or current from the pulses flows through, and thus is “capture” by the electrical energy of for example a pulse of electrical energy.

By the terms “charge” or “charged” or “polarity” or the like terms, when used in reference to electrodes are used interchangeably.

Current, I, (capital “i” of the Latin or Roman alphabet) is measured in ampere also known as amps.

The term “electrical current” and “current”, when with reference to electricity or electrical matters, are equivalents herein and are used interchangeably. When knowing the voltage and measuring the current to that pulse of electrical energy, or its electrical current, then impedance can be calculated from Ohm's Law equation.

By the term “epicardial” fat layer or pad or the like, this is used to mean the epicardial tissue of the body, for example human body. These terms are used interchangeably as are equivalents.

By the term “fat” as used herein this is used to describe a type of tissue, this is used to mean, for example, adipose tissue. The terms “fat” and “adipose” are used herein as interchangeable terms and means the same.

By the term “optimal voltage” or “suitable voltage” or the like, this is not necessarily the optimal, or optimum, voltage and the person skilled in the art would know that this term may include its use as a relative term. The term as used herein includes a voltage, or voltages, that balances the need for a high enough voltage, of an electrical energy to penetrate most of the adipose tissue layer to be effective at ablation, with the need to not be too great a voltage that the electrical field produced penetrate through the adipose tissue and may risk damage to non-targeted tissue, for example underlying muscle tissue.

The term “n” as used herein, is the number of the pulse, the term “N” as used herein, is the number of pulses—the total or greatest number, the greatest n value of a set of pulses is the last pulse n emitted, and the total number N of pulses.

By the term “the plurality of pulses comprising increasing voltage”, or the like phrase, as used herein means that each consecutive pulse has a voltage greater than the voltage value of the previous pulse, for example of a plurality of pulses.

By the term “previous” when used for pulses this means earlier in time and when the term is used as “the previous pulse emitted” or the like, this is used to describe the immediate earlier pulse emitted.

By the term “pulse” and the like, as used herein, this is used to mean a pulse of electrical energy, and includes, for example, a pulse of electrical energy emitted from an electrode.

Impedance, Z, or Resistance R is measured in ohms (Q). In the broader electrical technology field, Impedance and Resistance values may differ slightly in some situations. However as the invention herein described is only concerned with relative values, and uses pulses of a very short duration, for example 50 microseconds or less, and for comparison of voltage or Impedance/Resistance or current values, the pulse duration will remain constant, the terms “Impedance” and “Resistance” as used herein are used interchangeably having equivalent meaning.

By the term “rate of change of impedance” as used herein this is used to mean the rate of change of impedance value in relation to, or in response to, the voltage value. As used herein the voltage value is a known value that may change, for example increase with consecutive pulses and the subsequent current is measured to calculate the impedance. The rate of change of impedance, as used herein, is the acceleration or deceleration (the rate) of the impedance value, not the actual magnitude of the value. The rate of change can be seen as the slope or gradient of the line or curve from plotting impedance values on the y axis calculated from voltage values on the x axis of a graph.

Voltage V is voltage value, and measures in volts. Vn is the voltage for the pulse number n. By the term “known voltage” this term is used to mean the voltage set by the apparatus or used for a pulse. The term includes where the processor knows the voltage value, to display or use for calculations of other parameters.

By the term “thickness” of adipose layer or fat layer, or the like is used to mean the thickness of the fat layer, for example, the epicardial fat layer or epicardial fat pad, measured from the heart muscle tissue outwards, in direction, from the heart, or vice versa. The thickness of this layer may change around the heart. The term includes the distance from the electrodes, when place on a surface or side of the tissue, to the furthest point of the tissue away from the electrode in a perpendicular direction from the electrode. For example, before reaching the underlying muscle tissue. Again, a relative thickness value is adequate for the invention to work.

Any one or more features of any example, embodiment or aspect of the invention as herein described can be combined with any one or more features of any other example, embodiment or aspect of the invention.

Figures: Examples of working the invention, and examples of the invention will be described with reference to the drawings:

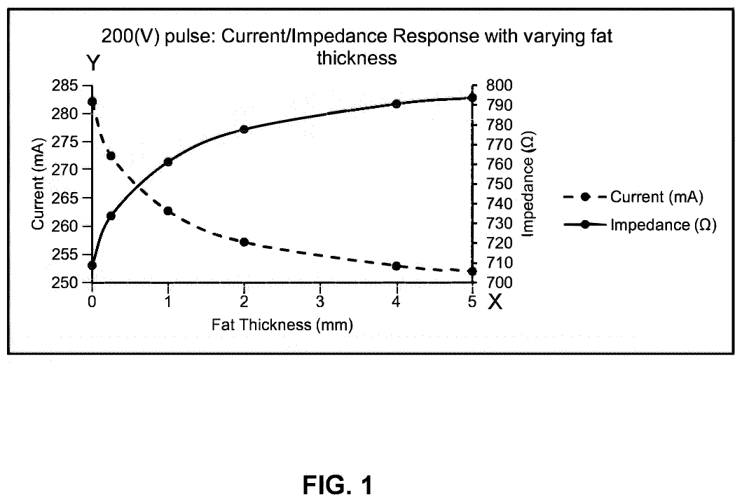

FIG. 1 shows a graph with increasing fat/adipose tissue thickness, and current and impedance, with a pulse of electrical energy of 200 volts.

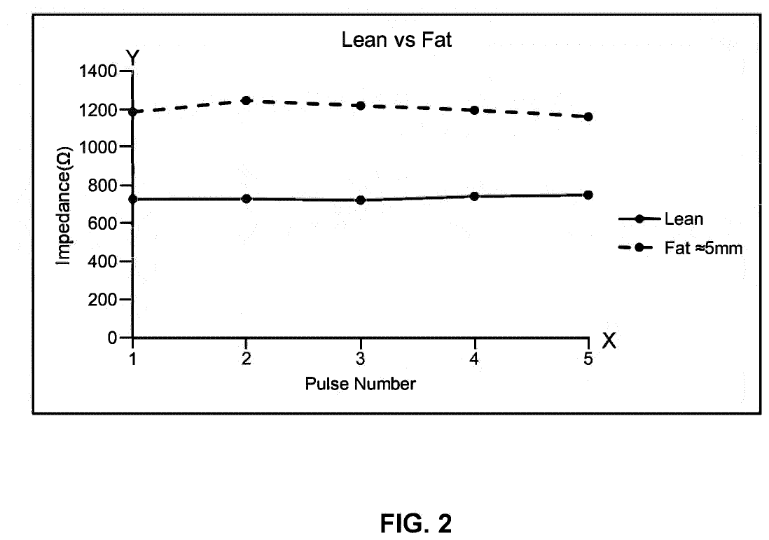

FIG. 2 shows a graph with Impedance values for fat and lean e.g. muscle tissue.

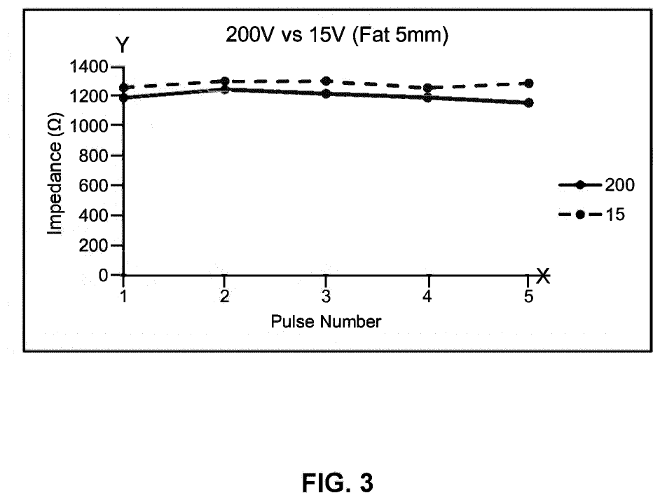

FIG. 3 shows Impedance values with pulses of two different voltages.

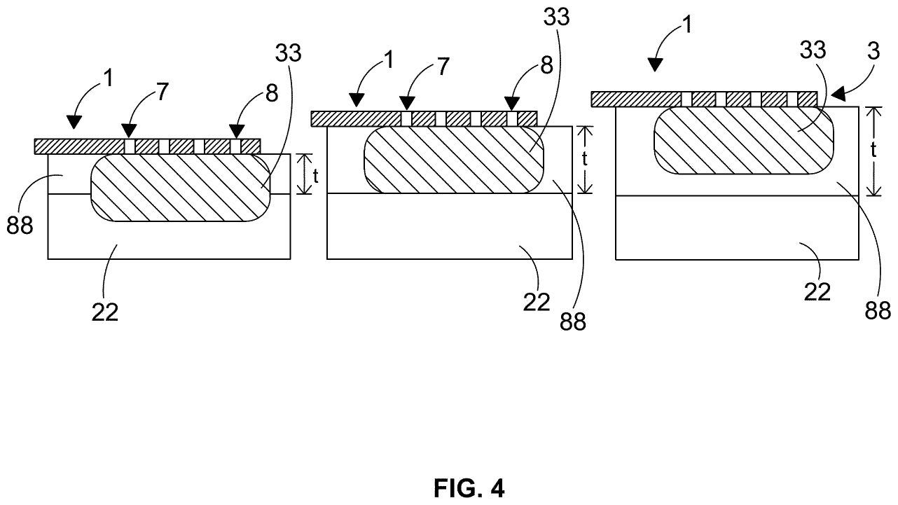

FIG. 4 shows the electric field within adipose tissue of different thicknesses.

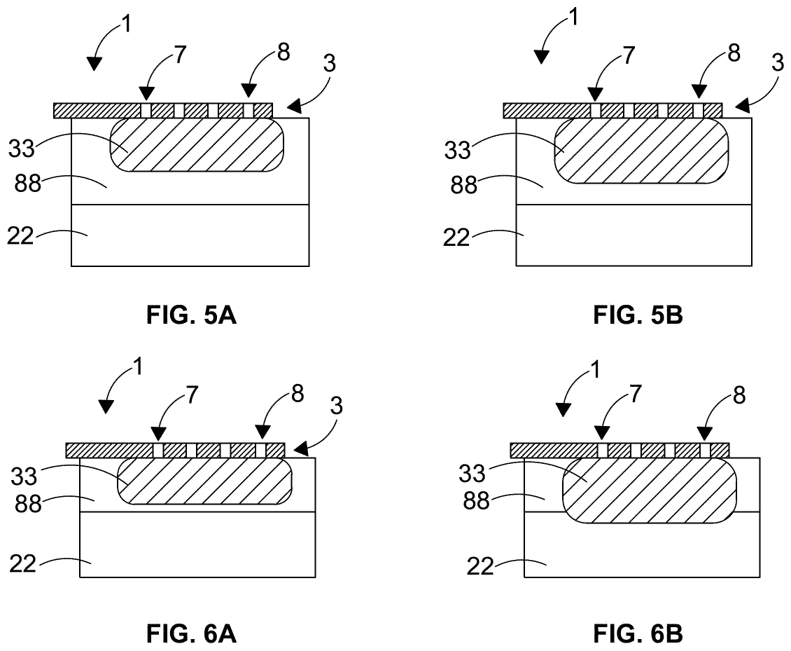

FIG. 5a and FIG. 5b shows the electric field of a 900 volt pulse and a 1000 volt pulse, within tissue.

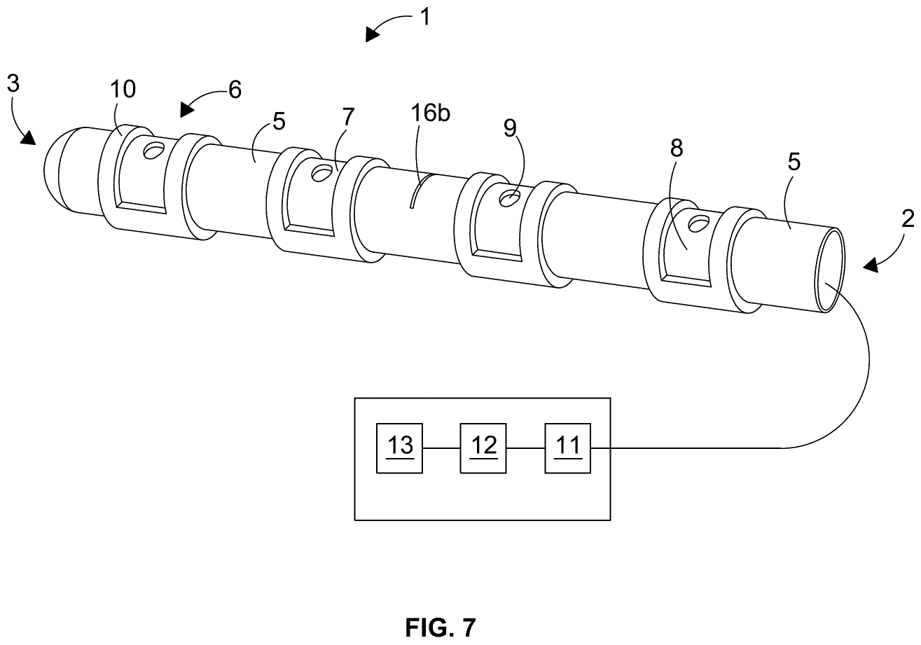

FIG. 6a and FIG. 6b shows the electric field of a 900 volt pulse and 1000 volt pulse, within tissue.

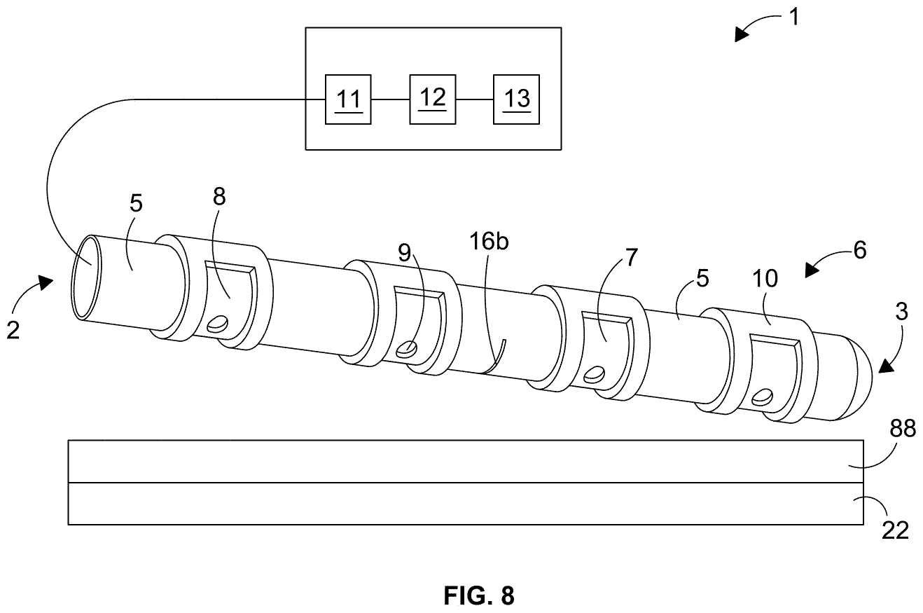

FIG. 7 shows a catheter suitable for use with the present invention.

FIG. 8 shows an embodiment of the present invention in use with tissue.

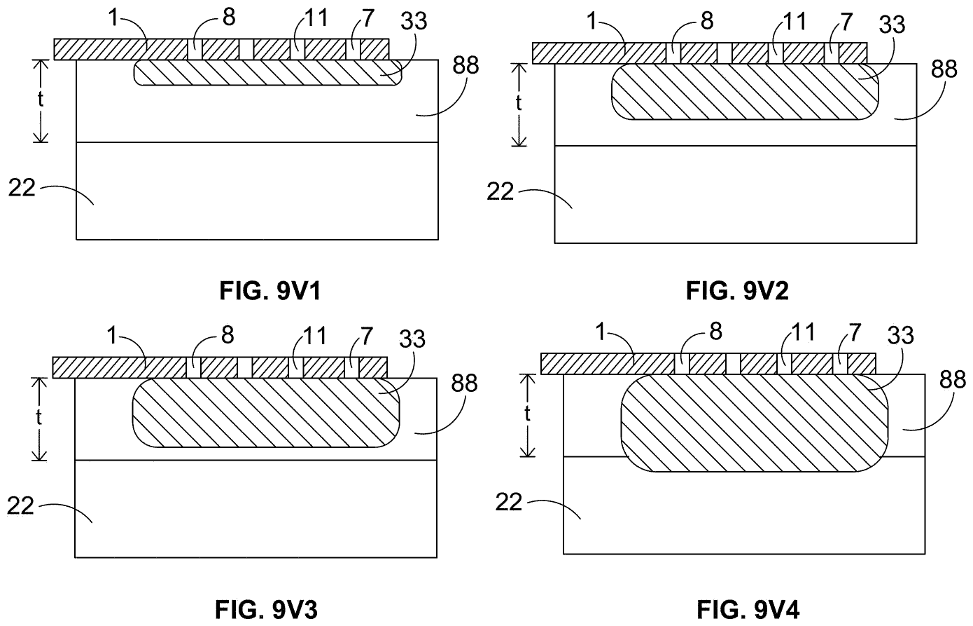

FIGS. 9, V1 to V4 shows increasing voltage values and the electrical field;

FIG. 10 shows a graph of the four impedance to voltage values of FIG. 9 1V to 4V.

FIG. 1 entitled 200 volts pulse: Current/Impedance Response with varying fat thickness, shows two graphs, generated from electric field models, comparing current in milli-Amps (mA) and impedance in Ohms both on the X axis compared with an increasing fat or adipose thickness in millimetres (mm) on the Y axis. On the X-axis, current is shown in the range between 250 and 285 mA and the impedance in ohms (Ω) is shown from 700 to 800 ohms Q. The fat thickness ranges from 0 to 5 millimetres.

| Fat thickness | 0 mm | 0.25 mm | 1 mm | 2 mm | 4 mm | 5 mm |
|---|---|---|---|---|---|---|
| Current mA | 282.5 | 272.5 | 262.5 | 257.5 | 253 | 252 |
| approx. | | | | | | |
| Impedance Ohms | 709 | 734 | 760 | 777 | 790 | 792 |
| approx. | | | | | | |

The electric field model was based on using pulses of electrical energy having 200 volts and a pulses duration of 100 microseconds, the electrical current could be measured and the Impedance Z could be calculated using Ohm's Law equation: Voltage equals Current times Impedance. V=I×Z.

From the model, it is predicted that the impedance value in ohms for an increasing thickness of fat tissue will increase, but that it will almost plateau, in that initially the impedance value increases quite quickly as fat thickness increases but then the rate of increasing impedance slows down, with increasing fat thickness. There is not much impedance difference once the thickness gets “too fat” maybe greater than 4 millimetres.

From the model, it is predicted that the current will decrease in value as the fat thickness increase. However, it is also predicted that the rate of decreasing current will also decrease as thickness of the fat increases, thus once the fat layer is greater than a certain amount, for example 4 millimetres, the change in current is small compared to the rate of change of current at lesser fat thickness, for example between 1 and 2 millimetres.

This is interesting as it shows the possible difficulty presented of measuring fat layers that are of a great thickness, for example 4 millimetres or greater, especially being able to measure differences between two layers of fat that are both thick.

FIG. 2 shows an example of data from experiments, which shows a very notable impedance difference when testing on lean cardiac tissue and on cardiac tissue with 5 mm of epicardial fat. In this example, a 200 V pulse with a duration of 100 microseconds us was applied. Good repeatability was also demonstrated, for example, with up to 5 identical pulses applied.

FIG. 2 shows the graph with Impedance on the X-axis ranging from 0 ohms to 1400 ohms and the pulse number along the Y-axis ranging from 1 to 5 pulses. The pulse of electrical energy was 200 volts with a duration of 100 microseconds, the fat thickness was constant at 5 millimetres in thickness. The electrodes, one of a positive polarity and one electrode of a negative polarity, spaced apart are placed on an upper surface of the fat layer and the electrical current that travels through at least a portion of the fat layer is measured and the impedance value calculated. The values are relative to this experiment but the general concept can be shown and repeated. A significant feature of the present invention is that the concept can use relative values of current and impedance to determine the voltage for ablation of an adipose tissue. The present invention can use the relative values of current and impedance to determining an optimal voltage for efficient ablation of the adipose tissue measured.

| Pulse No. | 1 | 2 | 3 | 4 | 5 |
|---|---|---|---|---|---|
| Impedance | 750 | 750 | 750 | 777 | 777 |
| Lean | | | | | |
| approx. | | | | | |
| Impedance | 1199 | 1250 | 1201 | 1199 | 1197 |
| Fat 5 mm | | | | | |
| approx. | | | | | |

The experiment as shown in graph 2/FIG. 2 shows that lean tissue for example muscle has a less impedance value compared to fat adipose tissue. Therefore, the pericardial adipose tissue around the heart has an impedance value greater than the underlying heart muscle tissue, in relative terms.

FIG. 3 repeats the experiment of FIG. 2 using a pulse of a different voltage, 15 volts, the duration of the pulse was the same. The 200 volt pulse results are included on the graph for comparison. In this example, however it is noted that the lower setting of 15V gives a marginally higher impedance in all repeat tests. It is likely that lower voltage does not allow the electric field to penetrate fully through the fat layer and is not capturing or the electric field is not extending to, any of the (lower Impedance) cardiac tissue underneath. Hence a higher value is obtained that may not be fully representative. Therefore, higher pulse voltages are preferred-ideally, so that they are high enough to fully penetrate through the most common range of fat thicknesses. Clinically, in the past at least, there may be reasons to limit the voltage and so 1000 V was likely to be a practical maximum. One, to two, 50 μs or 100 μs pulses at this voltage is unlikely to cause irreversible electroporation to cardiac tissue. The present invention however, may indicate where a higher voltage pulse may be used, that may have a low risk of penetrating completing through the adipose layer to other tissue.

| Pulse No. | 1 | 2 | 3 | 4 | 5 |
|---|---|---|---|---|---|
| Impedance | 1250 | 1250 | 1250 | 1250 | 1250 |
| Ohms approx | | | | | |

FIG. 4 shows a catheter 1 with a distal end 3 with four electrodes. In this example shown there are, two pairs, of electrodes, two electrodes are anodes 7 and two are cathodes 8, however in different examples this pairing, or charge or polarity, arrangement of the electrodes 7, 8, or the number of electrodes, may be different. Respectively for each of the three figures in FIG. 4, the catheter 1 is positioned adjacent to a different thickness of fat layer 88 with underlying cardiac muscle tissue 22. The catheter 1 is place in electrical contact or communication, with the fat layer 88, directly or indirectly. Thus, the electrical energy is emitted from the electrode, for example, in this example, DC pulsed electrical energy, and an electric field is generated that extends into at least a portion of the fat layer tissue 88. A salt solution may be used, in embodiments of the invention to ensure electrical communication or contact of the electrodes of the invention and the adipose or fat layer 88. The thickness t of the fat layers 88 increasing left to right of the three layers of fat 88 shown in FIG. 4. The adipose or fat tissue or layer 88 may in some embodiments comprises epicardial Ganglionated Plexi cells (GP).

FIG. 4 schematically shows the scenarios if delivering a 1000 V pulse, into tissue with different thickness t of adipose or fat layers 88. The middle case may show where that 1000V pulse is just enough so that the electric field 33 fully captures the fat 88—giving a nominal Impedance (Z). The left case may show that with a lower fat thickness t, we can expect to get a lower impedance value Z as some of the electric field 33 will be capturing the lower impedance cardiac tissue 22 under the fat 88. On the other hand, the right case shows what happens for a thicker layer of fat 88—here the 1000V penetrates no further than the adipose or fat layer 88 and so the response in terms of impedance is relatively unchanged from the middle scenario. This approach therefore presents the problems faced of determining a thickness of a fat layer as it is difficult to differentiate fat thickness above a threshold. Table 4 below 1 shows this data for two bipolar arrangements. This shows that at lower thickness values there is good differentiation between the impedance values obtained but at greater thickness of the fat layer there is less difference in the impedance values thus interpretation is more difficult at greater thickness values of a fat, or adipose, layer.

| Cases | Total Current (mA) | Impedance (Ω) |
|---|---|---|
| BP- 0.25 mm | 2381.0 | 419.99 |
| BP- 1 mm | 2027.4 | 493.24 |
| BP- 2 mm | 1850.5 | 540.39 |
| BP- 4 mm | 1841.0 | 543.18 |
| BP- 5 mm | 1832.8 | 545.61 |
| BP2- 0.25 mm | 3408.4 | 293.39 |
| BP2- 1 mm | 2823.4 | 354.18 |
| BP2- 2 mm | 2604.8 | 383.91 |
| BP2- 4 mm | 2591.2 | 385.92 |
| BP2-5 mm | 2587.0 | 386.55 |

BP1: electrode 1 and 2 paired against electrodes 3 and 4;
BP2: electrodes 2 and 3 paired against electrodes 1 and 4;

Electrode 1 is nearest to the catheter distal end, and the others are number consecutively from the distal end.

FIGS. 5a and 5b. In an embodiment of the present invention two different pulse voltages can be used to understand, or measure, if the full thickness t of the fat layer 88 is being captured, that the electric field 33 generated from the pulse of electrical energy extends the full thickness t of the fat layer 88. For example, an initial Impedance value is calculated for a 900 volt pulse, from the scenario as shown in FIG. 5a and a second Impedance value is calculated for a 1000 volt pulse from the scenario as shown in FIG. 5b. The electrical current value I is measured, using a current sensor, not shown in FIG. 5a or 5B but shown in FIG. 7. A catheter 1 with oppositely charged electrodes 7, 8 is to be positioned adjacent to, or in electrical contact with, the same thickness t of a fat layer 88. Similar to the methods described before, but here the thickness t of the fat layer 88 is constant and the voltage of the pulse of electrical energy is changed. The electric field 33 of the 1000 volt pulse, FIG. 5b will be greater in size than the electric field 33 of the lesser voltage, 900 volt pulse, FIG. 5a.

If there is no difference in the readings of the impedance value; or if there is a increase in the second impedance value with an increased voltage (compared to the previous pulse of lower voltage), this would suggest that the full thickness t of the fat 88 was not captured by the electric field 33 of the first pulse of electric energy, that the voltage of the first pulse of electrical energy maybe was not great enough to extend the full thickness t of the adipose or fat layer 88 and therefore not a voltage perhaps of optimal efficiency to ablate the adipose or fat layer 88 of that particular thickness t. It indicates that a higher voltage electrical energy pulse may perhaps be used to capture more of the adipose tissue layer without penetrating all the way through the adipose layer and capturing another tissue, for example, underlying muscle. The reason may be that the fat layer 88 thickness t is the same, or that the increased electrical field 33 is capturing just more of the fat/adipose layer 88, as seen in FIG. 5b, hence resulting in, maybe an increase in the Impedance value Z for the greater voltage pulse FIG. 5b. The actual values can be relative as just comparing the impedance values Z of the first lower voltage pulse with the second slightly higher voltage pulse. In alternative embodiments the apparatus may be configured to detect a rate of change impedance in relation to voltage, thereby indicating the interface between two tissues, the adipose tissue and muscle tissue as the rate of change of impedance in relation to voltage will decrease when the pulse of electrical energy encompasses muscle tissue. In alternative embodiments, the order of the pulse may be different, for example, a higher voltage pulse may be used before a lower voltage pulse. Likewise more than two different voltage pulses may be used, or more than one pulse at a particular voltage. The benefit of starting with the lower voltage pulse and increasing the voltage is that there may be less risk of subjecting underlying tissue, for example heart muscle, to the adipose layer 88 with electrical energy, or at least reduces the amount of high voltage electrical energy being subjected to the targeted or non-adipose tissue, for example muscle tissue.

In this example, but shown in FIG. 7, the emitted pulses may be controlled by a controller 12 and a processor 13 may calculate an impedance value from the measured electrical current measured from a current sensor 11. The current and electric field 33 will pass through at least a portion of the adipose tissue layer 88 being measured. The fat/adipose layer 88 is over and adjacent to a muscle layer 22, as reflects the usual situation of epicardial fat pad over the heart muscle tissue. The processor 13 is configured that the processor 13 with the controller 12 via the electrodes can emit the desired number of pulses of known voltage, and calculate the impedance very quickly, for example less than a second and thus can obtain information quickly to emit further pulses of electrical energy of different, for example, increasing voltage to repeat this again. This process can be repeated as necessary. The processor 13, current sensor 11, and controller 12, are configured to be able to repeat this process, including emitting pulse of known voltage, measuring the electrical current, and calculating an impedance value, and comparing the impedance value to previous impedance values, as is necessary to obtain a voltage for ablation of this tissue. For example, when the impedance value decreases for a pulse of greater voltage compared to a previous pulse of lesser voltage, then the previous voltage, the voltage of the previous pulse, may be a voltage for ablation of the adipose tissue measured. The voltage of the previous pulse may be an optimal voltage for ablation of the adipose tissue. In balancing capturing most of the adipose tissue and lessening the risk of penetrating through the adipose layer to no adipose tissue, for example muscle tissue.

To explain in more detail, if the second pulse of a higher voltage resulted in an impedance value that was less than the impedance value measured from the previous pulse of a lower voltage this would indicate that previous pulse did capture all, or substantially all the fat layer 88 in its generated electrical field 33 and not much, if any, of the underlying muscle tissue 22. This is because the muscle tissue 22 has a much lower impedance value compare to fat or adipose tissue 88, and it would appear that the electric field 33 of the pulse of higher voltage, but with the reduce impedance value, extended through the entire thickness of the fat layer 88 and extended into the underlying muscle tissue, to overall give a lower impedance value compared to the impedance value of the previous pulse. Thus, the determined voltage to use for ablation of this fat tissue 88 is the voltage of the previous pulse, or as in this example also the first pulse, of electrical energy. The voltage of the previous pulse would likely to have an electrical field 33 that did not extend to the underlying adjacent muscle tissue 22, that its electrical field 88 did not penetrate all the way through the thickness t of the adipose layer 88 to reach the muscle tissue 22 below, or at least did so at a lower extent than the electrical field 88 of the higher voltage pulse. Thus, the voltage of the previous pulse of electrical energy may present less risk to the patient, and potential less damage to the muscle tissue 22. This can be fine-tuned by have small incremental increasing in the voltage value, for example, increasing in steps of 1 or 2 volts at a time. The processor can process each pulse and its comparison very quickly, for example, less than 1 second, and therefore the whole process of determining the voltage for ablation of the adipose tissue can be completed very quickly too, for example less than a second.

Thus to explain in a hypothetical example, if the first pulse had a voltage of 900 volts and the pulse indicated a higher impedance value compared to a second pulse of electrical energy of 1000 volts, the 900 volt pulse would potentially be a less risk voltage to use for ablation of that thickness of fat layer 88. This is determined by relative values and not necessarily requiring the exact thickness t of the adipose layer 88 to be known or calculated. Thus, the invention may determine a voltage for subjecting to an adipose layer. The invention may determine an optimal voltage for ablating the particular layer of fat 88. In comparison to other voltages. The skilled person will understand that the value is relative but still effective to work the invention, or to give an effective voltage for ablation with less risk of damaging non-adipose tissue. The invention includes in vitro application.

FIG. 6 shows a further embodiment of the present invention wherein the, multiple sensing-pulses approach, can be further refined to improve the resolution/accuracy at which the transition from fat to tissue is detected. For example, more than two pulses can be applied and the voltage difference between the pulses can, for example, be made smaller, for example differing by only 10 volts. The collected data can be rapidly analyzed to identify which voltage is fully capturing the fat and therefore which voltage is likely to be needed for effective treatment.

Not shown in FIG. 6 (but shown in FIG. 7), there is a catheter 1 with electrodes 7, 8 of different polarity. There is also in electrical communication with the electrodes 7, 8, a current sensor 11 configured to measure the electrical current from any pulse of electrical energy, a controller 12 configured to actuate the electrode or electrodes to emit pulses of electrical energy of a desired known voltage; and a processor 13 configured to calculate an impedance value from the known voltage and current value of the electrical current from a pulse of electrical energy and also configured to compare the data from one pulse to another and if need be to vary the voltage of additional pulses of electrical energy and if need to stop increasing the voltage value of pulses when a voltage for ablation is determined. In different embodiments, the processor may be configured to have different additional or alternative functions.

FIG. 6a shows, a hypothetical example of an electrical field 33, from a 900 volt that almost extends to the full thickness t of the adipose layer 88, and does not penetrate any of the muscle layer 22 underneath the adipose or fatty layer 88. The adipose or fat tissue or layer 88 may in some embodiments comprises epicardial Ganglionated Plexi cells (GP). FIG. 6 b shows a consecutive or further pulse of electrical energy of 1000 volts and it can be seen that the electrical field 33, in this example, extends through the layer of fat 88 and reaches the underlying muscle tissue 22. Therefore the processor should detect that the impedance value for the 1000 volt pulse has decreased, or is lower than the impedance value for the 900 volt pulse, as the electrical field has penetrated to the muscle tissue 22, and thus the determined voltage value for ablation of this particular fat tissue is the voltage of the previous pulses, thus 900 volts. The 900 volts pulse may present less risk to a patient. The 900 volt pulse, in this example, with voltage increase steps of 100 volts, may potentially cause less damage to the muscle tissue underlying the fat tissue that requires ablation treatment. This example has, with voltage value step increases comprising 100 volts, determined a voltage, for example 900 volts, for ablation of the adipose layer. The actual thickness t of the adipose layer 88 is not required as the values are relative, but advantageously the invention can obtain real time information of the tissue for ablation to determine a, for example suitable, voltage for ablation. This may include a voltage for ablation that may potentially be less harmful than other greater voltages. This may include a voltage that is more effective than lesser valued voltage pulses, that potentially cannot penetrate enough of the fat layer.

FIG. 7 shows a catheter 1 suitable for use with the present invention. In electrical communication with the electrodes are a current sensor 11, a controller 12 and a processor 13. The controller is configured, to control the emitting of pulses from the electrodes. The current sensor is configured, to measure the electrical current from the electrical energy from the electrodes and communicate this information to the processor. The processor is configured, to receive data from the current sensor and calculate the impedance value of the tissue to be measured from the known voltage of the electrical current and the measured electrical current, The processor is configure, to be in communication with the controller, able to receive information from the controller but also to actuate the controller to actuate the electrodes to emit electrical energy of the desired pulse duration and voltage, for example. The processor is also configured, or comprises a configuration, to process the data of the apparatus and determine a voltage for ablation of the fat layer 88. In this embodiment, there are 4 electrodes shown of positive 7, and negative charge 8. The distal end 3 is pointed or round in shape to aid insertion into the human body and to position in the region of the heart. The four openings 6 of the catheter 1 are generally rectangular in shape and each opening 6 is 2.7 millimetre in length in the longitudinal direction of the catheter 1 and 2 millimetres in width across the longitudinal axis of the catheter. Alternative examples may have other sized and shaped openings. Alternative embodiments may have different size and shaped openings. Optionally, each opening 6 in this example, has a raised portion 10 surrounding the opening 6 and in this embodiment, the raised portion 10 extends around the catheter surrounding the opening 6. The raised portion 10 protrudes 1.2 millimetre from the wall 5 of the shaft of the catheter 1. The raised portion 10 of the catheter helps enables insulation to be positioned inside the catheter in the internal areas within the catheter 1, adjacent to the raised portions 10 on the inside surface of the raised portions 10 of the catheter. The insulation (not shown) is configured that it provides insulation against the electric field but still enables any wires and cables to pass along the internal conduit 4 (not shown as within wall or walls 5) of the catheter 1. There is no insulation blocking the electrodes 7 at the openings 6, thus there is no insulation adjacent to the electrodes 7 on the surface of the electrode that corresponds to the corresponding opening 6. In this embodiment, the electrodes protrude through the openings 6 by 1 millimetre from, or beyond, the wall 5 of the shaft of the catheter 1, in a radial direction from the axis of the catheter 1. In this embodiment, the catheter 1 also comprises an orientation mark 16a towards the proximal end 2 of the catheter 1 on the wall 5 of the shaft of the catheter 1. The orientation mark 16a is a visible line indicating the direction that the electrodes 7 face. The orientation mark 16a is a line that runs partially along the longitudinal length of the wall 5 of the catheter. In this embodiment, the catheter also comprises an orientation mark 16b that is a radiopaque mark that is largely a semi-circular line that largely crosses the longitudinal direction of the catheter 1. The orientation mark 16b also indicates the electrode 7 facing direction, and with the aid of X-Rays or similar like radiation and imaging equipment, the electrode face orientation can be seen on a screen thus the desired positioning of the catheter 1 can be achieved. In the present example, the orientation mark 16b is shown towards the distal end of the catheter 1. However, in alternative embodiments that have an orientation mark 16b, the orientation mark may be positioned elsewhere on the catheter 1 for example, between the most distal electrode 7 and the distal end of the catheter 1. In this embodiment, the catheter comprises plastic. The catheter may assist in positioning the electrodes in the desires location even for in vitro application of the invention. Also shown in the FIG. 7 embodiment are holes 9 in the electrodes. Other embodiments may not have these option holes 9.

FIG. 8 shows another embodiment of the present invention for use with tissue 88, 22. In this embodiment the catheters 7 and 8 are held within a catheter 1 for ease of holding or positioning the electrodes 7, 8, although other embodiments may not have or require a catheter. This particular FIG. 8 embodiment shows the catheter 1 with openings 6 in the wall 5 of the shaft of the catheter 1, however other embodiments of catheters for the invention may not have these. The catheter 1 has a distal end 3. The electrodes 7 and 8 are spaced apart by the catheter 1. This FIG. 8 embodiment has optional raised portions 8 of the catheter, but other catheters 1 may not have these in other embodiments. Also present in this embodiment is a sensor 11 for measuring the current of each and every pulse of electrical energy emitted, and a controller 12 and processor 13 in electrical communication with each other and the senor 11 and the electrodes 7, 8. The controller 12 and processor 13 are configured to be able to calculate the impedance value of each pulse of electrical energy and/or the rate of change of the impedance in relation to voltage for pulses of electrical energy. In use the electrodes 7, 8 would be positioned adjacent the tissue 88, 22 preferable adjacent the adipose 88 tissue of the adipose muscle tissue 88, 22, such that the adjacent tissue 88, 22 completes the electrical circuit between the electrodes 7 and 8, thus electrical energy with travel through at least a portion of the adjacent tissue 88, 22. The apparatus embodiment shown may calculate a suitable or optimal voltage to use for subjecting the adipose tissue, or adjacent adipose tissue 88 when in use, to a voltage for ablation, of for example target cells within the adipose tissue 88, without, or minimising the electrical field extending beyond the adipose layer to the muscle tissue 22. Excessive electrical energy to the muscle tissue 22 can be harmful to that muscle tissue causing death of the muscle tissue 22. Also shown in the FIG. 8 embodiment are holes 9 in the electrodes. Other embodiments may not have these option holes 9. The holes 9 may allow dispensing of saline. Also shown in the FIG. 8 embodiment is an optional orientation mark 16b, as explained above.

FIGS. 9 V1 to FIG. 9 V4 show an embodiment of the present invention with pulses of electrical energy with increasing voltage. FIG. 9 V4 shows a greater voltage value of emitted pulse of electricity than the voltage value of the emitted pulse shown in V3, which has a greater voltage value of emitted pulse than shown in V2 which has a greater voltage value emitted pulse as shown in V1. In this embodiment the electrode 7 emits the pulse of voltage creating the field of electrical energy. The electrical energy passes through the adjacent tissue 88, 22 to reach the electrode 8 of opposite charge to electrode 7. The sensor 11 measures the current of the and each emitted pulse of electrical energy. A controller 12 and/processor 13 (not shown in FIG. 9) may process the information and the controller 12 and/or processor 13 are able to react to the collected information and calculated information to give an output. In this example the controller 12 and the processor 13 are in electronic communication with the sensor and electrodes. The electrodes 7 and 8 are spaced apart but are configured that when placed adjacent a tissue, the tissue completes the electric circuit between the two electrodes and electrical energy 33 may travel from one electrode 7 to the other electrode 8. The adjacent tissue is made up of adipose tissue 88 and muscle tissue 22. In some embodiments the adjacent tissue may be cadaver tissue. In this example the electrodes 7 and 8 and the sensor 11 are held within a catheter 1 for ease of positioning the electrodes 7, 8 and sensor 11. The adipose tissue has a thickness t. The exact value of t is not required. As can be seen the emitted pulses of electrical energy in V1, V2 and V3, that are increasing in turn in voltage value, create an electrical field 33 that although is increasing in size with increased voltage value, does not reach or encompass the muscle tissue 22. For this illustrative purpose the exact value of the voltage of the emitted pulses of electrical energy is not required. Of the 4 example voltage values shown it is only the greatest value V4 that has a voltage value great enough that creates an electrical field large enough to encompass a portion of the muscle tissue 22. What is happening here in V4 is that some electrical energy is passing through a portion of the muscle tissue. In some embodiments the impedance value may drop as electrical current takes the easiest route of travel and impedance is less when the electrical energy travels through muscle tissue 22 compared to the adipose tissue 88. However in some embodiments as shown in this embodiment it is the rate of change of the impedance value that decreases, in relation to the voltage that indicates when the electrical field 33 has encompassed the muscle tissue 22. Thus the desired voltage to use for ablation is a lesser voltage than used for V4, as one would prefer a voltage that does not encompass muscle tissue 22, or a voltage that does not extend beyond the adipose tissue 88 to be subjected to ablation. (even if the actual target cells are not the actual adipose cells but other cells within the adipose tissue.) The safer voltage value to use therefore is the voltage value of the V3 emitted pulse of electrical energy, as this is the voltage value that creates an electrical field that covers or penetrates or treats, more of the adipose tissue 88 than V1 or V2 but does not encompass or extend into muscle tissue 22 like the voltage value of V4. The decrease in the rate of change of impedance in relation to voltage can be seen in the graph of FIG. 10, wherein the value or curve produced with the V4 reading of the rate of change drops or decreases compared to the rate of change of impedance calculated before for the V1, V2 and V3 voltage values. The steepness of the curve is seen to drop with the V4 reading indicating a drop or decrease in the rate of change of impedance in relation to the voltage. As mentioned for illustrative purposes in this shown embodiment the actual values of voltage used are not required, but the controller and processor can work out the voltage of the emitted pulse used prior to and especially immediately prior to the voltage that showed a decrease in the rate of change of impedance or showed a drop in the curve or slop of the graph recording the rate of change of impedance may be a suitable, or optimal voltage to use for subjecting the adjacent adipose to a voltage for ablation. This suitable voltage may be one that encompasses or penetrate more of the adipose tissue than other voltages but does not extend beyond the adipose tissue to penetrate muscle tissue which may be underlying the adipose tissue. The most optimal voltage lies between the V3 voltage used and the V4 voltage. The V4 voltage being too high as it is likely to produce an electrical filed that will penetrate the muscle tissue 22. It can be foreseen that the smaller the incremental increase in the voltage value for consecutive pulses of electrical energy, the more accurate the determined suitable or optimal voltage may be. With very small increase in voltage value it may be possible to determine a voltage value that produces an electrical field 33 that covers all, or very near all, the adipose tissue 88 without extending into the muscle tissue 22.

FIG. 10 shows the impedance values plotted on a graph with voltage V along the x axis and impedance R along the y axis. The impedance value calculated from the measured current of the pulse of electrical energy. The rate of change of the impedance in relation to voltage is the acceleration or deceleration of the change and can be seen here as an acceleration, positive, the slope or gradient, is increasing. However it may be seen that the V4 value although higher in total impedance has a less steep curve (lesser slope or gradient) and thus the rate of change of impedance in relation to voltage has decreased compared to the rate of change of impedance in relation to voltage, of the V3 value and pulse of electrical energy. In this example shown the rate of change of impedance in relation to voltage appears to be constant for the V2 and V3 pulses of electrical energy, as the curve is straight, the rate is not increasing or decreasing although the actual impedance value is increasing with increased voltage. Other embodiments may have different shaped graphs, but the invention is able to detect the change and therefore detect the change is tissue between for example adipose tissue and the underlying muscle tissue. The present invention is able to do this without exact values of impedance required or exact thicknesses of the tissue to determine a safe suitable voltage for use for ablation to subject the adjacent tissue to.

Aspects of the invention will be described by way of non-limiting numbered examples:
1. An apparatus for determining a voltage of electrical energy for subjecting to an adipose tissue layer, comprising:
   at least a first electrode, and at least a second electrode, wherein the at least first electrode and the at least second electrode are spaced apart from each other, and wherein the at least first electrode and the at least second electrode comprise different polarity;
   a controller configured for actuating at least a first electrode to emit a pulse of known voltage of electrical energy; and
   a current sensor configured for measuring an electrical current, from the emitted pulse of electrical energy emitted from the said electrode and which said pulse extends through a portion of an adjacent tissue; and,
   a processor configured for; calculating an impedance value, from the known voltage of the pulse of emitted electrical energy and the electrical current; and for determining a voltage for subjecting to an adjacent tissue layer by comparing the calculated impedance value of an adjacent tissue with one or more previous calculated impedance values.
2. An apparatus as described in example 1 wherein the current sensor is configured for measuring an electrical current, from the emitted pulse of electrical energy emitted from said electrode and configured such that the said pulse extends through a portion of an adjacent tissue wherein the adjacent tissue comprises adipose tissue.
3. An apparatus as described in either example 1 or 2, wherein the controller is further configured for actuating emitting of at least a first pulse of electrical energy of a known voltage, from any one or more of the following group: the at least first electrode; the at least second electrode, and any subsequent electrode.
4. An apparatus as described in any one of examples 1 to 3, wherein the controller is further configured for actuating the at least first electrode to emit at least a first pulse of electrical energy, and at least a second pulse of electrical energy, wherein the at least first pulse of electrical energy and the at least second pulse of electrical energy are of different voltage.
5. An apparatus as described in any preceding example 1 to 4 wherein the controller is further configured for actuating the said electrode to emit at least a second pulse of electrical energy comprising a greater voltage value than the voltage value of at least a first pulse of electrical energy.
6. An apparatus as described in any preceding example 1 to 5 wherein the controller is further configured for actuating the electrode to emit a plurality of consecutive pulses of electrical energy wherein the number of the plurality of consecutive pulses is n; and, each pulse has a known voltage V.
7. An apparatus as described in example 6, wherein each consecutive pulse increasing in number n has an increasing voltage V value.
8. An apparatus as described in anyone of examples 1 to 7, wherein the current sensor is further configured for measuring the electrical current of each pulse n; and, the processer is configured for calculating an impedance value Z, from the known voltage V, and the electrical current I for each pulse n; and to monitor the impedance values; and to compare the impedance value Z for each pulse n with the impedance value to, an, or the, earlier emitted pulse.
9. An apparatus as described in example 8 wherein, the processor is further configured for, to signal the controller upon detection that, the impedance value Z of the pulse n, decreases, or is less than, in value compared to the impedance value Z of an, or the earlier emitted pulse.
10. An apparatus as described in either one of examples 8 or 9, wherein the processor is configured for, to signal the controller, or a user, that the voltage for subjecting to an adipose layer, is the voltage V of the earlier pulse.
11. An apparatus as described in any one of examples 9 or 10, wherein the controller is configured for, that when signalled that the impedance Z for a pulse from the plurality of consecutive pulses, has decreased in value, or is less than, compared to the impedance value with a, or the, previous pulse, the controller is configured to stop increasing the voltage of the pulses of electrical energy to be emitted.
12. An apparatus as described in any one of examples 9 to 11 wherein the controller is further configured for, that when signalled that the impedance Z of a pulse from the plurality of pulses, has decreased in value, or is less than, compared to the impedance value of a, or the previous pulse, the controller is configured to indicate to a user that the apparatus is ready for ablation mode.
13. An apparatus as described in any one of examples 9 to 12, wherein the controller is further configured for, that when signalled that the impedance Z has decreased in value, or is less than in value to that determined for a previous pulse from the plurality of pulses comprising increasing voltage, the controller is configured, to emit electrical energy via the at least one electrode to ablate tissue at the determined voltage, for subjecting to an adipose layer or for subjecting to the adjacent tissue.
14. An apparatus as described in any previous example 1 to 13 wherein the pulsed electrical energy comprises pulses between 10 and 3100 volts.
15. An apparatus as described in any previous example 1 to 13 wherein the duration of the pulsed electrical energy comprises between 10 and 200 milliseconds.

1. An apparatus for determining a voltage of electrical energy for subjecting to an adipose tissue layer for ablation, comprising:
at least a first electrode, and at least a second electrode, wherein the at least first electrode and the at least second electrode are spaced apart from each other, and wherein the at least first electrode and the at least second electrode comprise different polarity;
a controller configured for actuating at least a first electrode to emit a plurality of pulses of known voltage of electrical energy;
a current sensor configured for measuring an electrical current, for each emitted pulse of electrical energy emitted from the said electrode when said pulse extends through a portion of an adjacent tissue completing the electrical circuit; and
a processor configured for calculating an impedance value, from the known voltage of the said pulse of emitted electrical energy and the measured electrical current; and for determining a voltage for subjecting to an adjacent tissue layer for ablation, by comparing the calculated impedance value of a pulse of electrical energy of a known voltage with one or more previous calculated impedance values calculated from previously emitted pulses of electrical energy of a different voltage value.

2. The apparatus of claim 1 wherein the current sensor is configured for measuring an electrical current, from each of the emitted plurality of pulses of electrical energy emitted from said electrode and configured such that the said pulse extends through a portion of an adjacent tissue wherein the adjacent tissue comprises adipose tissue.

3. The apparatus of claim 1, wherein the controller is further configured for actuating emitting of at least a first pulse of electrical energy of a known voltage, from any one or more of the following group: the at least first electrode; the at least second electrode, and any subsequent electrode.

4. The apparatus of claim 1, wherein the controller is further configured for actuating the at least first electrode to emit at least a first pulse of electrical energy, and at least a second pulse of electrical energy, wherein the at least first pulse of electrical energy and the at least second pulse of electrical energy are of different voltage and wherein the voltage value of the at least second said pulse is greater than the voltage value of the at least first or earlier said pulse.

5. The apparatus of claim 1, wherein the controller is further configured for actuating the electrode to emit a plurality of consecutive pulses of electrical energy wherein the number of the plurality of consecutive pulses is n; and, each pulse has a known voltage V, and wherein the voltage value increases for consecutive pulses of electrical energy, for at least a desired period of time, or for a desired number of consecutive pulses or until a predetermined voltage has been reached or until manually stopped, or until the controller stops any further change of voltage

6. The apparatus of claim 5, wherein each consecutive pulse increasing in number n has an increasing voltage V value of 0.1 volts.

7. The apparatus of claim 1, wherein the processer is configured for calculating an impedance value Z, from the known voltage V of the said pulse, and the measured electrical current I for each said pulse n; and to monitor the impedance values; and to compare the impedance value Z for each pulse n with the impedance value to, an, or the, earlier emitted pulse.

8. The apparatus of claim 1, wherein the processor is configured to calculate the rate of change of the impedance value in relation to the voltage for the pulses of electrical energy.

9. The apparatus of claim 1, wherein the processor is further configured for, to signal the controller, or user, or both user and controller, upon detection that, the impedance value Z of the pulse n, decreases, or is less than, in value compared to the impedance value Z of an, or the earlier emitted pulse.

10. The apparatus of claim 1, wherein the processor is further configured for, to signal the controller, or user, or both controller and user, upon detection that, the rate of change of the impedance value of the said pulse n compared to the change of the voltage values, decreases, or is less than, in 5 value compared to the rate of change of the impedance value of an, or the earlier emitted pulse.

11. The apparatus of claim 9, wherein the processor is configured for, to signal the controller, or a user, or both a user and controller, that the voltage for subjecting to an adjacent, for example adipose, layer for ablation, is the voltage V of the earlier emitted pulse.

12. The apparatus of claim 9, wherein the controller is configured for, that when signalled that the impedance Z for a pulse from the plurality of consecutive pulses, decreases in value, or is less than in value, compared to the impedance value with a, or the, previous emitted pulse, or the rate of change of the impedance value compared to the change of the voltage, decreases, or is less than in value to previously calculated rate of change of impedance value, the controller is configured to stop increasing the voltage value of the pulses of electrical energy to be emitted.

13. The apparatus of claim 9, wherein the controller is further configured for, that when signalled that the impedance Z of a pulse from the plurality of pulses, decreases in value, or is less than, compared to the impedance value of a, or the previous emitted pulse, or the rate of change of the impedance value compare to the change of voltage, of pulses, decreases, or is less in value compared to previous values of the rate of change of impedance, the controller is configured to indicate to a user, or the controller, or both the controller and a user, that the apparatus is ready for ablation mode.

14. The apparatus of claim 9, wherein the controller is further configured for, that when signalled that the impedance Z has decreased in value, or is less than in value to that determined for a previous emitted pulse from the plurality of pulses comprising increasing voltage, or the rate of change of impedance value in relation to the change of voltage of pulses, decreases compare to previous calculated rate of change values of impedance for previously emitted said pulses the controller is configured, to emit electrical energy via at least one electrode to ablate tissue at the determined voltage, for subjecting to an adipose layer or for subjecting to the adjacent tissue.

15. The apparatus of claim 1, wherein the pulsed electrical energy comprises pulses between 10 and 3100 volts.

16. The apparatus of claim 1, wherein the duration of the pulsed electrical energy comprises between 10 and 200 milliseconds.
